(19) United States
(12) Patent Application Publication
(10) Pub. No.: US 2004/0101527 A1
(43) Pub. Date: May 27, 2004

(54) CD18-binding antibodies and use thereof for inhibition and alleviation of stenosis-related symptoms and disorders

(75) Inventor: Christopher J. Horvath, Taunton, MA (US)

Correspondence Address: FISH & RICHARDSON PC, 225 FRANKLIN ST, BOSTON, MA, 02110, US

(73) Assignee: Millennium Pharmaceuticals, Inc., a Delaware corporation

(21) Appl. No.: 10/716,028

(22) Filed: Nov. 17, 2003

Related U.S. Application Data
(63) Continuation of application No. 09/531,088, filed on Mar. 18, 2000, now abandoned.

(51) Int. Cl. A61K 39/395

(52) U.S. Cl. 424/144.1

(57) ABSTRACT

The invention is based on the discovery that molecules (e.g. a monoclonal antibody or a portion thereof) which bind specifically with the CD18 subunit of a CD18-containing leukocyte cell-surface antigens (e.g. a cell-surface antigen such as Mac-1 which contains both CD18 and a form of CD11) can be used to inhibit, prevent, and alleviate vascular stenotic and restenotic lesions and symptoms and disorders associated with such lesions.

### CROSS-REFERENCE TO RELATED APPLICATIONS

Not Applicable.

### STATEMENT REGARDING FEDERALLY SUPPORTED RESEARCH OR DEVELOPMENT

Not Applicable.

### BACKGROUND OF THE INVENTION

The present invention relates generally to compositions and methods for inhibiting, preventing, and alleviating stenosis and restenosis and symptoms and disorders associated with stenosis and restenosis.

A relatively common complication of surgical and catheter-mediated vascular interventions is stenosis or restenosis of a portion of the blood vessel. For example, arterial stenosis can occur at or near the site of vascular grafting, such as within coronary arteries following coronary bypass surgery. Restenosis remains the principal complication limiting the long-term efficacy of percutaneous transluminal coronary angioplasty and other balloon angioplasty procedures. Similarly, placement of stents within the arterial lumen can induce stenosis at the placement site.

Vascular stenoses are associated with migration of smooth muscle cells to the site of the stenosis, proliferation of smooth muscle cells at that site, or both. However, the factor(s) that are responsible for inducing or sustaining migration and proliferation of smooth muscle cells are not yet known with certainty. It is known that local accumulation of platelets and leukocytes (e.g. neutrophils) occurs at sites of vascular injury, beginning shortly after occurrence of the injury and continuing at least several days thereafter. Although leukocyte activation is associated with restenosis, at least in humans, it has been found by others that administration of agents which inhibit inflammation (e.g. glucocorticoids) fails to inhibit restenosis in humans (Pepine et al., 1990, Circulation 81:1753-1761; Pietersma et al., 1995 Circulation 91:1320-1325; Mickelson et al., 1996, J. Amer. Coll. Cardiol. 28:345-353; Inoue et al., 1996, J. Amer. Coll. Cardiol. 28:1127-1133). Despite the severe need for them, pharmaceutical agents which are relatively broadly efficacious to prevent, inhibit, or alleviate stenotic and restenotic conditions have been difficult to identify, and only a few such agents have been described.

It is known that antibodies which are capable of inhibiting binding between the leukocyte cell-surface antigen designated Mac-1 (also known as CD11b/CD18 or αMβ2) and one of its ligands (e.g. ICAM-1 or ICAM-2) enhance vascular healing and lessen stenosis and restenosis of blood vessels following injury (International patent application publication number WO 98/42360). However, others previously taught that monoclonal antibodies that bind specifically to the CD18 portion of Mac-1 are ineffective for preventing, inhibiting, or alleviating restenosis, and that such antibodies may actually worsen the symptoms of restenosis (Guzman et al., 1995, Coron. Art. Dis. 6:693-701). Others who commented on the effect of monoclonal antibodies (designated 60.3 and R15.7) which were asserted to specifically bind with the CD18 portion of leukocyte cell-surface antigens did not examine what, if any effect, such antibodies might have on development or progression of stenosis and related symptoms and disorders in humans (Kling et al., 1992, Arterioscler. Thromb. 12:997-1007; Kling et al., 1995, Circ. Res. 77:1121-1128; Golino et al., 1997, Thromb. Haemost. 77:783-788). Indeed, the rabbit model used in these studies by others has been demonstrated not to be predictive of anti-stenotic efficacy in humans. Results obtained using primate models of human stenoses have been more consistent with results obtained in humans. By way of example, heparin is ineffective to prevent restenosis in humans and other primates, but has been shown to inhibit or prevent restenosis in other experimental systems, including experimental mammals.

The present invention satisfies a long-felt need for pharmaceutical agents which are efficacious for inhibiting, preventing, and alleviating stenotic and restenotic lesions and symptoms associated with them.

### BRIEF SUMMARY OF THE INVENTION

The invention relates to a method of inhibiting stenosis (i.e. including restenosis) in a human blood vessel. This method comprises administering to the human an anti-CD18 antibody which binds specifically with at least the CD18 portion of a mammalian (e.g. human) protein which comprises CD18. Stenosis is thereby inhibited in the vessel.

In this method, the anti-CD18 antibody can, for example, be an antibody which binds specifically with substantially only the CD18 portion of the protein, an antibody that has an epitopic specificity which is the same as or similar to that of monoclonal antibody 1B4, or monoclonal antibody 1B4. In one embodiment of the method, the anti-CD18 antibody binds specifically with at least the CD18 portion of a primate protein which comprises CD18.

The mammalian protein with which the anti-CD18 antibody binds can, for example, be a leukocyte cell-surface antigen, such as one of Mac-1, LFA-1, p150,95, and CD11d/CD18. Mac-1 is a preferred antigen. In one aspect, binding of the anti-CD18 antibody with the antigen inhibits binding of a natural ligand of the antigen therewith, such as one of ICAM-1, ICAM-2, ICAM-3, C3bi, factor X, fibrin, and fibrinogen (i.e. one of ICAM-1, C3bi, factor X, fibrin, and fibrinogen for Mac-1). Binding of the anti-CD18 antibody with the protein can modulate one or more functions normally associated with binding of a natural ligand of the protein therewith. By way of example, the function can be one selected from the group consisting of binding of leukocytes with vascular endothelium, translocation of leukocytes through vascular endothelium, infiltration of leukocytes into intimal vascular tissue, release of a chemotactic factor from leukocytes in a vascular tissue, release of a growth factor from leukocytes in a vascular tissue, leukocyte-binding-associated release of a chemotactic factor from a vascular tissue, and leukocyte-binding-associated release of a growth factor from a vascular tissue. The leukocyte can, for example, be a neutrophil.

In one aspect, this method is used to inhibit stenosis in a blood vessel in which the vascular endothelium has been traumatically perturbed. For example, the blood vessel can be one of a grafted blood vessel, a blood vessel in which an angioplasty balloon has been inflated, a blood vessel comprising a portion at which a laser angioplasty procedure has been performed, a blood vessel which has sustained a crushing injury, and a blood vessel into which a stent has been placed. Of course, the blood vessel can also be a vessel in which the vascular endothelium has non-traumatically deteriorated, such as an atherosclerotic blood vessel and an arteriosclerotic blood vessel. The blood vessel can, for example, be a coronary blood vessel or a cerebral blood vessel.

The anti-CD18 antibody used in this method can, for example, be a whole antibody, an antibody fragment (e.g. one of a Fv, Fab, Fab′, or F(abN)₂ fragment), a chimeric antibody, a humanized antibody, or a fully human antibody.

The anti-CD18 antibody can, according to this method, be administered to the human by providing the anti-CD18 antibody to the blood vessel (e.g. prior to traumatically perturbing the endothelium of the vessel, during such traumatic perturbation, or after traumatically perturbing the endothelium of the vessel). The perturbation can, for example, comprise an angioplastic intervention (e.g. a balloon angioplastic intervention or emplacement of a vascular stent within the vessel).

Substantially the same method can be used to alleviate an existing or developing stenosis in a human blood vessel.

The invention further relates to a kit for assessing stenosis in a human blood vessel. The kit comprises an anti-CD18 antibody having a detectable label (e.g. a gamma radiation source) and an instructional material which describes detecting the anti-CD18 antibody in a blood vessel of the human.

The invention includes another method of inhibiting or alleviating stenosis in a human blood vessel. This method comprises removing leukocytes which bind specifically with an anti-CD18 antibody from the human's blood. Stenosis is thereby inhibited or alleviated in the vessel

Also included in the invention is a method of inhibiting interaction of a leukocyte having a CD18-containing cell-surface protein with vascular endothelium in a human. This method comprises contacting the leukocyte with an anti-CD18 antibody. Interaction of the leukocyte with vascular endothelium is thereby inhibited. The leukocyte can, for example, be selected from the group consisting of lymphocytes, monocytes, granulocytes, neutrophils, T cells, and basophils. The interaction that is inhibited can, for example, be binding of the leukocyte with the vascular endothelium or translocation of the leukocyte across the vascular endothelium.

The invention further includes a method of assessing the presence of leukocytes associated with vascular stenosis in blood obtained from a human. This method comprises

a) contacting the blood with an anti-CD18 antibody and

b) detecting binding of the anti-CD18 antibody with leukocytes in the blood.

Binding of the anti-CD18 antibody with leukocytes in the blood is an indication of the presence of leukocytes associated with vascular stenosis in the blood. Binding of the anti-CD18 antibody with leukocytes in the blood can be assessed qualitatively, or it can be quantified (e.g. by comparing binding of the anti-CD18 antibody with leukocytes in the blood of the human with binding of the anti-CD18 antibody with leukocytes in control blood obtained from a human who is either:

i) afflicted with vascular stenosis or

ii) not afflicted with vascular stenosis.

The invention still further includes a kit for assessing the presence of leukocytes associated with vascular stenosis in blood obtained from a human. This kit comprises

i) an anti-CD18 antibody and

ii) an instructional material which describes at least one of

a) quantifying the presence of the leukocytes in the blood of the human,

b) the content of the leukocyte in the blood of a human afflicted with vascular stenosis, and

c) the content of the leukocyte in the blood of a human not afflicted with vascular stenosis.

In another aspect, the invention relates to a method of inhibiting a disorder associated with stenosis in a blood vessel of a human. This method comprises administering to the human an anti-CD18 antibody which binds specifically with at least the CD18 portion of a mammalian protein which comprises CD18. Stenosis is thereby inhibited in the vessel, and the disorder is thereby inhibited.

In yet another aspect, the invention relates to a method of alleviating a disorder associated with stenosis in a blood vessel of a human. This method comprises administering to the human an anti-CD18 antibody which binds specifically with at least the CD18 portion of a mammalian protein which comprises CD18. Stenosis is thereby alleviated in the vessel, and the disorder is thereby alleviated.

### BRIEF DESCRIPTION OF THE DRAWINGS

FIG. 1 is a graph which depicts monoclonal antibody concentration in serum obtained from test animals, as assessed by ELISA.

FIG. 2, comprising FIGS. 2A and 2B, is a pair of graphs which depict the degree of leukocyte target saturation in the presence of an anti-CD18 antibody (triangles) or in the presence of a control monoclonal antibody (circles). FIG. 2A depicts the degree of neutrophil target site saturation, and FIG. 2B depicts the degree of monocyte target site saturation.

FIG. 3, comprising FIGS. 3A, 3B, 3C, and 3D, is a quartet of graphs which depict leukocyte counts in animals to which monoclonal antibody 1B4 (triangles) or a control monoclonal antibody (SS1; circles) had been administered. Total white blood cell counts are shown in FIG. 3A; total neutrophil counts are shown in FIG. 3B; total lymphocyte counts are shown in FIG. 3C; total monocyte counts are shown in FIG. 3D.

FIG. 4 is a graph which indicates mean anti-monoclonal antibody titers in animals to which monoclonal antibody 1B4 (triangles) or a control monoclonal antibody (circles) had been administered.

FIG. 5, comprising FIGS. 5A, 5B, and 5C, depict blinded quantitative angiography results obtained in test animals, as described herein in the Example. FIG. 5A is a graph which shows luminal diameter as a function of time in animals to which monoclonal antibody. 1B4 (squares) or a control monoclonal antibody (circles) had been administered. FIG. 5B and 5C are bar graphs which show the difference between late luminal loss and the index LLL/ALG in animals to which monoclonal antibody 1B4 or a control monoclonal antibody had been administered.

FIG. 6, comprising FIGS. 6A and 6B, is a pair of bar graphs which depict the results of histomorphometric analysis of intimal area (in square millimeters; FIG. 6A) and the ratio of intima to media in blood vessels obtained from animals to which either monoclonal antibody 1B4 or a control monoclonal antibody had been administered and which had undergone balloon angioplasty (“Balloon Only”) or both balloon angioplasty and endovascular stent emplacement (“Balloon and Stent”).

FIG. 7, comprising FIGS. 7A and 7B, is a pair of images which depict the difference between blood vessels obtained from animals to which either monoclonal antibody 1B4 or a control monoclonal antibody had been administered.

### DETAILED DESCRIPTION OF THE INVENTION

The invention is based on the discovery that, contrary to teachings in the prior art (e.g. Guzman et al., 1995, Coron. Art. Dis. 6:693-701), molecules which bind specifically with the CD18 subunit of the Mac-1 leukocyte cell-surface antigen can be used to inhibit, prevent, and alleviate vascular (e.g. arterial) stenotic and restenotic lesions and symptoms (e.g. ischemia) and disorders (e.g. vascular graft rejection) associated with such lesions. It is recognized that CD18-binding antibodies can bind with CD18 when it is complexed with CD11b (i.e. as in the Mac-1 antigen), when it is complexed with CD11a (i.e. as in the LFA-1 antigen), when it is complexed with CD11c (i.e. as in the p150,95 antigen), and when it is complexed with CD11d.

The present invention relates to an antibody or a functional fragment thereof which specifically binds with a mammalian (preferably primate, and more preferably human) CD18 protein, such as is found in human leukocyte cell-surface antigens designated Mac-1, LFA-1, p150,95, and CD11d/CD18. As used herein, an “anti-CD18 antibody” is used to refer collectively and individually to whole antibodies and to fragments of such whole antibodies wherein the antibodies or fragments exhibit specific binding with CD18 protein. In one embodiment, the anti-CD18 antibody binds specifically with CD18 protein, but does not bind specifically with any other component of antigens Mac-1, LFA-1, p150,95, and CD11d/CD18 (e.g. the anti-CD18 antibody does not specifically bind with any of the CD11 subunits of these antigens). Although substantially any antibody which binds specifically with CD18 can be used, it is preferred that the anti-CD18 antibody be one which binds specifically regardless of whether CD18 is complexed with another protein in a leukocyte cell-surface antigen, such as a CD11 subunit (e.g. one of CD11a, CD11b, CD11c, and CD11d).

In one embodiment, the anti-CD18 antibody is raised against an isolated mammalian (e.g. human) CD18 protein or an isolated CD18-containing protein (e.g. a leukocyte cell-surface antigen). Of course the anti-CD18 antibody can, alternatively, be raised against a recombinant mammalian CD18 or a portion thereof (e.g. a polypeptide comprising an epitope which is normally exposed on CD18 protein, particularly one which is normally exposed when the protein is complexed with a CD11 subunit in a leukocyte membrane). The anti-CD18 antibody can furthermore be raised against a host cell (e.g. a human leukocyte such as a neutrophil or a bacterial cell which has been transformed to express CD18) which expresses mammalian CD18. As used herein “raising” an antibody against a target can be accomplished using any known method. Examples of methods used to raise antibodies against a target include providing the target to the body (e.g. to the bloodstream) of a vertebrate (e.g. a rabbit, hamster, mouse, kangaroo, goat, sheep, pig, or cow), isolating immune cells from the vertebrate, selecting one or more immune cells which produce an anti-CD18 antibody, and obtaining anti-CD18 antibodies from that cell or cells. An alternative example is to combine the target with a library (e.g. a filamentous phage library, such as an M13 phage library) of particles (cells, phage, vectors, etc.) which have an antibody or a fragment of an antibody exposed on their surface and which contain or can be correlated with a nucleic acid encoding the exposed antibody or fragment, select one or more particles which bind with the target, obtain the nucleic acid contained within or correlated with the particle, and use the obtained nucleic acid to generate or design an anti-CD18 antibody.

In a preferred embodiment, the antibody specifically binds human CD18 protein or a portion thereof, and in a particularly preferred embodiment the antibody has specificity for a naturally occurring or endogenous human CD18 protein. Anti-CD18 antibodies which can inhibit one or more functions characteristic of a mammalian CD18 protein are encompassed by the present invention. Examples of these functions include mediating binding of leukocytes with endothelial cells, binding of leukocytes with cells having ICAM-1 protein at the surface thereof, binding of leukocytes with cells having ICAM-2 protein at the surface thereof, mediating translocation of leukocytes across an endothelium, mediating movement of leukocytes into diseased, damaged, or inflamed tissue, and mediating release of chemotactic or growth modulating substances (e.g. growth factors and chemokines) from leukocytes and from cells with which leukocytes bind. For example, in one aspect, an anti-CD18 antibody inhibits (i.e. reduces the frequency or strength of or prevents) interaction of CD18 with a natural ligand, such as ICAM-1 or ICAM-2.

The epitope of CD18 with which the anti-CD18 antibody binds is not critical. In one embodiment, the anti-CD18 antibody binds with the same epitope with which monoclonal antibody 1B4 binds. Monoclonal antibody 1B4 is described, for example in European Patent Application publication number EP 0438312A2. Preferably, the anti-CD18 antibody binds with an epitope of CD18 that is different from the epitope(s) with which previously known anti-CD18 antibodies (e.g. antibodies R15.7 and 60.3) bind or, when the anti-CD18 antibody binds with the same epitope of CD18 as a known anti-CD18 antibody, binds with a greater affinity than the known antibody.

The anti-CD18 antibody of the present invention can inhibit functions mediated by human CD18, including recruitment of leukocytes to and into damaged or inflamed tissues, such as vascular tissue which has been subjected to a surgical or catheter-mediated intervention, such as balloon angioplasty. The antibody can inhibit such functions to an extent that generally relates to the concentration of the antibody in fluid at the corresponding body site. It is recognized that there is a concentration of the antibody above which further antibody will have no substantially greater inhibitory effect. At lower concentrations, the inhibitory effect of the antibody will vary with the concentration of the antibody in a manner which can be predicted by the skilled artisan using routine experimentation. Effective concentrations at which the anti-CD18 antibody described herein can be used include, for example, 1000, 500, 200, 100, 50, 20, 10, 5, 2, or 1 microgram per milliliter. It is understood that where delivery of the anti-CD18 antibody can be locally administered (e.g. within a discrete body compartment such as the synovial fluid of a joint), a lower concentration of antibody can be used than is possible where administered generally (e.g. systemic administration via the blood stream).

In a further embodiment of the invention, the anti-CD18 antibody can inhibit binding of CD18 with a ligand thereof (e.g. with ICAM-1 or ICAM-2), preferably with an IC₅₀ of less than about 50, 20, 10, 5, 2, 1, or (preferably) 0.5 micrograms per milliliter. In a further embodiment of the invention, the anti-CD18 antibody binds with CD18 with an affinity of at least about 10⁻⁷, 10⁻⁸, or (preferably) 10⁻⁹ molar.

A humanized mono clonal antibody which binds specifically with CD18 was produced as described herein, and is designated 1B4. In a preferred embodiment, the antibodies of the present invention bind human CD18, and have an epitopic specificity which is the same as or similar to that of the 1B4 antibody described herein. Antibodies with an epitopic specificity which is the same as or similar to that of 1B4 monoclonal antibody can be identified using art-recognized techniques. For example, antibodies having an epitopic specificity which is the same as or similar to that of 1B4 can be identified by their ability to compete with 1B4 monoclonal antibody for binding to human CD18 (e.g. to cells bearing human CD18, such as leukocytes bearing one or more of the Mac-1, LFA-1, p150,95, and CD11d/CD18 cell-surface antigens), by their ability to inhibit binding of 1B4 to human CD18, or using receptor chimeras (as described, for example in Rucker et al. 1996, Cell 87:437-446). Using these or other suitable techniques, antibodies having an epitopic specificity which is the same as or similar to that of an antibody of the present invention can be identified. The invention also relates to a bi-specific antibody, or functional fragment thereof (e.g. F(ab′)₂), which has the same or similar epitopic specificity as 1B4 and least one other antibody (see, e.g. U.S. Pat. No. 5,141,736 (Iwasa et al.), U.S. Pat. Nos. 4,444,878, 5,292,668, 5,523,210 (all to Paulus et al.) and U.S. Pat. No. 5,496,549 (Yamazaki et al.).

The anti-CD18 antibodies of the present invention can be polyclonal or monoclonal antibodies. Furthermore, it is understood that methods described herein which utilize anti-CD18 antibodies can be performed using whole anti-CD18 antibodies, CD18 antigen-binding fragments of whole anti-CD18 antibodies, monoclonal antibody 1B4, antibodies (i.e. whole antibodies and CD18-binding antigen fragments) which have the same or similar epitopic specificity as 1B4, and combinations of these. These antibodies and fragments can, optionally, be used in combination with antibodies or antibody fragments which do not specifically bind with CD18.

The anti-CD18 antibodies described herein can be used in therapeutic, diagnostic, preventive, prognostic, and research applications as described herein. The present invention encompasses an anti-CD18 antibody (e.g. monoclonal antibody 1B4, or a CD18-binding fragment thereof) for use in therapy (including prophylaxis) or diagnosis (e.g. of particular diseases or conditions as described herein), and use of such an antibody for manufacture of a medicament for use in treatment, prevention, or diagnosis of diseases or conditions as described herein.

Preparation of immunizing antigen, and production of polyclonal and monoclonal antibody can be performed as described herein, or using other suitable techniques. A variety of methods have been described (see e.g. Kohler et al., Nature, 256: 495-497 (1975) and Eur. J. Immunol. 6: 511-519(1976); Milstein et al., Nature 266: 550-552 (1977); Koprowski et al, U.S. Pat. No. 4,172,124; Harlow, E. and D. Lane, 1988, Antibodies: A Laboratory Manual, (Cold Spring Harbor Laboratory: Cold Spring Harbor, N.Y.); Current Protocols In Molecular Biology, Vol. 2 (Supplement 27, Summer '94), Ausubel, F. M. et al., Eds., (John Wiley & Sons: New York, N.Y.), Chapter 11, (1991)). Generally, a hybridoma can be produced by fusing a suitable immortal cell line (e.g. a myeloma cell line such as SP2/0) with an antibody-producing cell. The antibody-producing cell, preferably obtained from the spleen or from one or more lymph nodes, can obtained from an animal to which the antigen of interest has been provided, either alone or in combination with other agents (e.g. in combination with an adjuvant, or as a cell which has the antigen exposed on its surface). Fused cells (i.e. hybridomas) can be isolated using selective culture conditions, and cloned by limiting dilution. Cells which produce antibodies with the desired binding properties can be selected by a suitable assay (e.g. using an ELISA to detect CD18-binding antibodies).

Other suitable methods of producing or isolating antibodies which bind CD18, including human or artificial antibodies, can be used, including, for example, methods which select recombinant antibody (e.g. single chain Fv or Fab) from a library, or which rely upon immunization of transgenic animals (e.g. mice) capable of producing a repertoire of human or artificial antibodies (see e.g. Jakobovits et al., Proc. Natl. Acad Sci. USA, 90: 2551-2555 (1993); Jakobovits et al., Nature, 362: 255-258 (1993); Lonberg et al., U.S. Pat. No. 5,545,806; Surani et al., U.S. Pat. No. 5,545,807). By way of example, anti-CD18 antibodies can be generated by providing CD18 or a fraction thereof, optionally admixed with an adjuvant or encompassed within a chimeric protein, to a genetically engineered strain of mice in which mouse antibody gene expression is suppressed and functionally replaced with human antibody gene expression, such as the Xenomouse™ strain of mice available from Abgenix, Inc. (Freemont, Calif.), and then isolating anti-CD18 antibodies from the mice.

Single chain antibodies, and chimeric, humanized, human, or primatized (CDR-grafted) antibodies, as well as chimeric or CDR-grafted single chain antibodies, and the like, comprising portions derived from different species, are also encompassed by the present invention and the term “antibody”. The various portions of these antibodies can be joined together chemically by conventional techniques, or can be prepared as a contiguous protein using genetic engineering techniques. For example, nucleic acids encoding a chimeric, human, or humanized chain can be expressed to produce a contiguous protein. See, e.g. Cabilly et al., U.S. Pat. No. 4,816,567; Cabilly et al, European Patent number 0,125,023 B1; Boss et al., U.S. Pat. No. 4,816,397; Boss et al., European Patent number 0,120,694 B1; Neuberger, M. S. et al., WO 86/01533; Neuberger, M. S. et al., European Patent number 0,194,276 B1; Winter, U.S. Pat. No. 5,225,539; Winter, European Patent number 0,239,400 B1; and Queen et al., U.S. Pat. Nos. 5,585089, 5,698,761 and 5,698,762. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single chain antibodies.

In addition, functional fragments of antibodies, including fragments of chimeric, humanized, primatized, human (i.e. fully human), or single chain antibodies, can also be produced. Functional fragments of the foregoing antibodies retain at least one binding function and/or modulation function of the full-length antibody from which they are derived. Preferred functional fragments retain an antigen-binding function of a corresponding full-length antibody (e.g. retain the ability to bind a mammalian CD18). Particularly preferred functional fragments retain the ability to inhibit one or more functions characteristic of a mammalian CD18, such as ability to modulate binding of leukocytes with endothelium or trans-endothelial migration of leukocytes. For example, in one embodiment, a functional fragment can inhibit the binding of human leukocytes (e.g. neutrophils) with tissues (e.g. endothelia) which naturally comprise ICAM-1 or ICAM-2. In another embodiment, a functional fragment can inhibit extravasation of neutrophils into intimal vascular tissue at the site of an intravascular balloon angioplastic intervention. Other activities which fuctional fragments can modulate, in some embodiments, include leukocyte trafficking, T cell activation, inflammatory mediator release, and leukocyte degranulation.

For example, anti-CD18 antibody fragments capable of binding to a mammalian CD18 or a portion thereof, including Fv, Fab, Fab′ and F(abN)₂ antibody fragments, are encompassed by the invention. Such fragments can be produced by enzymatic cleavage of whole anti-CD18 antibodies or by recombinant techniques, for example. For instance, papain or pepsin cleavage can generate Fab or F(ab′)₂ fragments, respectively. Antibodies can also be produced in a variety of truncated forms using antibody genes in which one or more stop codons has been introduced upstream of the natural stop site. For example, a chimeric gene encoding a F(ab′)₂ heavy chain portion can be designed to include DNA sequences encoding the CH₁ domain and hinge region of the heavy chain.

By way of example, chimeric anti-CD18 antibodies (including fragments of whole anti-CD18 antibodies) can be prepared by synthesizing a DNA which comprises DNA segments of human (e.g. human constant regions) and non-human (e.g. murine complementarity-determining regions; CDRs) segments linked in a manner which encodes the chimeric antibody or fragment, and by then transcribing and translating the DNA to produce a contiguous polypeptide chain.

The terms “humanized immunoglobulin” and “humanized antibody” as used herein refer to an immunoglobulin, or a portion of an immunoglobulin, comprising portions of immunoglobulins of different animal origin, wherein at. least one portion is of human origin. Accordingly, the present invention relates to a humanized immunoglobulin which specifically binds with mammalian CD18 (e.g. human CD18 or Cynomolgus monkey CD18). This humanized antibody comprises an antigen-binding region of non-human (e.g. rodent) origin and at least a portion of an immunoglobulin of human origin (e.g. a human framework region, or one or more human constant regions or portions thereof). For example, the humanized antibody can comprise one or more portions derived from an immunoglobulin of non-human (e.g. murine) origin which exhibits the requisite anti-CD18 specificity one or more immunoglobulin portions of human origin, joined together chemically by conventional techniques (i.e. to yield a synthetic antibody) or prepared as a contiguous polypeptide using recombinant (i.e. ‘genetic engineering’) techniques. An example of a humanized immunoglobulin of the present invention is an immunoglobulin containing one or more immunoglobulin chains comprising a CDR of non-human origin (e.g. one or more CDRs derived from an antibody of non-human origin) and a framework region derived from a human light chain, a human heavy chain, or some hybrid thereof, (i.e. CDR-grafted antibodies, with or without framework changes).

In one embodiment, the humanized anti-CD18 antibody can compete with 1B4 monoclonal antibody for binding with human CD18. In a preferred embodiment, the antigen-binding region of the humanized immunoglobulin (a) is derived from 1B4 monoclonal antibody (e.g. as in a humanized immunoglobulin comprising CDR1, CDR2 and CDR3 of the 1B4 light chain and CDR1, CDR2 and CDR3 of the 1B4 heavy chain).

Humanized anti-CD18 antibodies can be produced using synthetic nucleic acids, recombinant nucleic acids, or both, to prepare genes (e.g. cDNA) encoding the desired humanized chain. For example, nucleic acid (e.g. DNA) sequences encoding humanized variable regions can be constructed using PCR mutagenesis methods to alter DNA sequences encoding a human or humanized chain, such as a DNA template from a previously humanized variable region (see e.g. Kamman, M., et al., NucL. Acids Res., 17: 5404 (1989)); Sato, K., et al., Cancer Research, 53: 851-856 (1993); Daugherty, B. L. et al., Nucleic Acids Res., 19(9): 2471-2476 (1991); and Lewis, A. P. and J. S. Crowe, Gene, 101: 297-302 (1991)). Using these or other suitable methods, variants can also be readily produced. In one embodiment, cloned variable regions can be mutagenized, and sequences encoding variants with the desired specificity can be selected (e.g. from a phage library; see e.g. Krebber et al., U.S. Pat No. 5,514,548; Hoogenboom et al., WO 93/06213, published Apr. 1, 1993; Knappik et al., WO 97/08320, published Mar. 6, 1997)) By “gene,” as used in this context is meant an expressible nucleic acid construct encoding a whole anti-CD18 antibody or a fragment thereof which binds specifically with CD18. Such a gene generally includes an translational start site and a translational stop codon. Where the gene is DNA, the gene should also include a transcriptional start site, and can further include a poly-adenosine region, as is known in the art.

Anti-idiotypic antibodies are also provided. Anti-idiotypic antibodies recognize antigenic determinants associated with the antigen-binding site of another antibody. Anti-idiotypic antibodies can be prepared against a second antibody by immunizing an animal of the same species, and preferably of the same strain, as the animal used to produce the second antibody, as described, for example, in U.S. Pat. No. 4,699,880.

The present invention also pertains to the 1B4 hybridoma cell line described herein, to the anti-CD18 monoclonal antibody produced by that hybridoma cell line, and to CD18-binding fragments thereof. This cell line, and other hybridomas which produce anti-CD18 antibodies, have uses beyond production of the monoclonal antibodies. For example, the cell lines can be fused with other cells (such as human myeloma, mouse myeloma, human-mouse heteromyeloma, or human lymphoblastoid cells, any of which can be made, or selected to be, sensitive to a selected drug using known methods) to produce additional hybridomas. These additional hybridomas can be used to transfer the genes encoding the monoclonal antibodies to a cell population or tissue, either in vitro or in vivo.

In addition, anti-CD18 antibody-producing cell lines can be used as a source of nucleic acids which encode one or more anti-CD18 antibody chains, and these nucleic acids can be isolated, expressed (e.g. upon transfer to other cells using any suitable technique (see e.g. Cabilly et al., U.S. Pat. No. 4,816,567; Winter, U.S. Pat. No. 5,225,539)), or otherwise manipulated (e.g. combined with portions of other antibody-encoding nucleic acids in order yield a nucleic acid encoding a chimeric anti-CD18 antibody). For instance, clones comprising a rearranged light or heavy chain of an anti-CD18 antibody can be isolated (e.g. by PCR) or. cDNA libraries can be prepared from mRNA isolated from the cell lines, and cDNA clones encoding an anti-CD18 immunoglobulin chain can be isolated. Thus, nucleic acids encoding a heavy chain, a light chain, or both, of an anti-CD18 antibody can be obtained and used in accordance with recombinant DNA techniques for production of specific immunoglobulins, immunoglobulin chains, or variants of either of these (e.g. to produce humanized immunoglobulins) in a variety of host cells or in an in vitro translation system.

Nucleic acids, encoding variants such as a human or humanized anti-CD18 antibody, or a heavy or light chain thereof, can be provided to a prokaryotic or eukaryotic vector (e.g. an expression vector such as a plasmid or a virus vector) and introduced into a host cell (e.g. by transformation, transfection, electroporation, or infection of the cell). Known expression control elements (e.g. promoters, terminator regions, transcriptional regulatory regions, and the like) can be included in the vector or integrated into the host cell genome in order to facilitate expression of the antibody or chain in the host cell. Production of the encoded chain(s) in the host cell can be achieved by maintaining the cells under conditions suitable for expression (e.g. in the presence of inducer, suitable media supplemented with appropriate salts, growth factors, antibiotic, nutritional supplements, etc.) as are known in the art. If desired, the encoded protein can be recovered and/or isolated from the host cells or the medium or tissue in which the host cells express the nucleic acids. This method of production encompasses expression of anti-CD18 antibodies or chains thereof in a host cell of a transgenic animal, as described, for example, in international publication WO 92/03918.

As described herein, anti-CD18 antibodies of the present invention can block (inhibit) binding of CD18 and a ligand thereof. Inhibition of CD18/ligand binding can thus be used to inhibit one or more functions associated with such binding. As discussed below various methods can be used to assess inhibition of binding of a ligand with CD18, and these methods can thus be used to assess inhibition of a function associated with CD18/ligand binding.

Binding Assays

As used herein “mammalian CD18” refers to naturally occurring or endogenous mammalian CD18 proteins and to proteins having an amino acid sequence which is the same as that of a naturally occurring or endogenous corresponding mammalian CD18 protein (e.g. recombinant proteins). Accordingly, as defined herein, the term includes mature CD18 protein, homomultimeric CD18 proteins, heteromeric proteins comprising at least one CD18 polypeptide (e.g. leukocyte cell-surface antigens Mac-1, LFA-1, p150,95, and CD11d/CD18, each of which comprises a {different} CD11 subunit and a CD18 subunit), polymorphic or allelic variants of these proteins, and other isoforms of these proteins (e.g. produced by alternative splicing or other cellular processes). Both post-translationally modified forms of the foregoing proteins and forms which are not so modified are included (e.g. glycosylated and non-glycosylated CD18 proteins and CD18-containing heteromers). Mammalian CD18 proteins can be isolated from natural sources or produced by recombinant or other synthetic methods. Naturally occurring or endogenous mammalian CD18 proteins include wild type proteins such as mature CD 18, polymorphic or allelic variants and other isoforms which occur naturally in mammals (e.g. humans and non-human primates), and heteromeric proteins which comprise CD18 (e.g. leukocyte cell-surface antigens such as Mac-1, LFA-1, p150,95, and CD11d/CD18). Such proteins can be recovered or isolated from a source which naturally produces mammalian CD18, for example. These proteins and mammalian CD18 proteins having the same amino acid sequence as a naturally occurring or endogenous corresponding mammalian CD18, are referred to by the name of the corresponding mammal. For example, where the corresponding mammal is a human, the protein is designated as a human CD18 protein (e.g. a recombinant human CD18 produced in a suitable host cell).

“Functional variants” of mammalian CD18 proteins include functional fragments, functional mutant proteins, and functional fusion proteins (e.g. those produced via mutagenesis or recombinant techniques). Generally, fragments or portions of mammalian CD18 proteins include those having a deletion (i.e. one or more deletions) of an amino acid residue (i.e. one or more amino acid residues) relative to the mature mammalian CD18 protein (such as amino-terminal, carboxyl-terminal, or internal deletions). Fragments or portions in which only contiguous amino acids have been deleted or in which non-contiguous amino acids have been deleted relative to mature mammalian CD18 protein are also envisioned.

Generally, mutants of mammalian CD18 proteins include natural or artificial variants of a mammalian CD18 protein differing by the addition, deletion, substitution, or some. combination of these, of one or more contiguous or non-contiguous amino acid residues. Such mutations can be in a conserved or non-conserved region, as assessed by inter-mammalian-species CD18 amino acid sequence homology.

Generally, fusion proteins encompass polypeptides comprising a mammalian CD18 (e.g. human CD18) or a variant thereof as a first moiety, linked via a peptide bond to a second moiety not occurring in the mammalian CD18 as found in nature. Thus, the second moiety can be an amino acid, oligopeptide or polypeptide. The first moiety can be in an amino-terminal location, a carboxyl-terminal location, or a location internal to the fusion protein. In one embodiment, the fusion protein comprises an affinity ligand (e.g. an enzyme, an antigen, epitope tag) as the first moiety, and a second moiety comprising a linker sequence and human CD18 or a portion thereof. Additional (e.g. third or fourth). moieties can also be present.

A “functional” fragment, portion, mutant, or fusion protein of a mammalian CD18 protein refers to an isolated, recombinant, or both, polypeptide which has at least one function characteristic of a mammalian CD18 protein, as described herein. Preferred functional variants can bind a ligand (e.g. one or both of ICAM-1 and ICAM-2), and are referred to herein as “ligand binding variants” of CD18.

In one embodiment, a functional variant of mammalian CD18 shares at least about 85% sequence identity with the corresponding mammalian CD18 (e.g. human CD18, as described in GenBank accession number NM_{—}000211, or another primate CD18), preferably at least about 90% sequence identity, and more preferably at least about 95% sequence identity with said mammalian CD18. In another embodiment, a functional fusion protein comprises a first moiety which shares at least about 85% sequence identity with the corresponding mammalian CD18, preferably at least about 90% sequence identity, and more preferably at least about 95% sequence identity with the mammalian CD18. Sequence identity can be determined using a suitable program, such as the Blastx program (Version 1.4), using appropriate parameters, such as default parameters set forth at the NCBI web site (http://www.ncbi.nlm.nih.gov/BLAST/). In one embodiment, parameters for Blastx search are scoring matrix BLOSUM62, W=3. In another embodiment, a functional variant comprises a nucleic acid which has a sequence which differs from the naturally-occurring nucleic acid molecule but which, due to the degeneracy of the genetic code, encodes mammalian CD18 or a portion or functional variant thereof.

A composition comprising an isolated or recombinant mammalian CD18 protein, or a functional variant thereof, can be maintained under conditions suitable for binding. Under such conditions, the protein is contacted with an antibody or an antibody fragment, and binding is detected, directly or indirectly. In one embodiment, cells which naturally express CD18 or cells comprising a recombinant nucleic acid sequence which encodes a mammalian CD18 or variant thereof are used. The cells are maintained under conditions appropriate for expression of the receptor. The cells are contacted with an antibody or fragment under conditions suitable for binding (e.g. in a suitable binding buffer), and binding is detected by standard techniques.

The extent of binding of a CD18 protein or variant with an antibody or antibody fragment can be determined relative to a suitable control. Comparison with a control can, for example, comprise assaying CD18 binding under a background condition which includes the absence of the antibody or fragment, or it can comprise comparing binding of CD18 with the antibody/fragment relative to binding of CD18 with a second antibody (e.g. a second antibody having a known affinity for CD18, such as monoclonal antibody 1B4). A cellular fraction comprising CD18 protein (e.g. a membrane fraction) or a synthetic composition (e.g. liposomes) comprising CD18 can be used in lieu of whole cells in the binding assay.

In one embodiment, an antibody or fragment is labeled with a suitable label (e.g. fluorescent label, isotope label, antigen or epitope label, enzyme label) prior to determining its ability to bind with CD18, and binding is determined by detection of the label. In another embodiment, bound antibody is detected using a labeled second antibody which specifically reacts with the antibody or fragment for which CD18 binding ability is being assessed. Specificity of binding can be assessed by competition or displacement, for example, using labeled or non-labeled antibody or a ligand as competitor, according to any of several known methods.

Binding inhibition assays can also be used to assess whether an antibody or fragment is an anti-CD18 antibody. In these assays, binding of the anti-CD18 antibody inhibits binding of another compound with CD 18. The other compound can be, for example, an anti-CD18 antibody of known specificity or a known ligand of CD18 (e.g. ICAM-1, ICAM-2, or a cell bearing either of these) or a functional variant of CD18. For example, a binding assay can be conducted in which binding of a known ligand of CD18 (e.g. monoclonal antibody 1B4) is compared with binding of an unknown or suspected ligand of CD18. Reduction in the extent of known ligand/CD18 binding is an indication that the unknown or suspected ligand binds specifically with CD18. A composition comprising an isolated or recombinant mammalian CD18 or a functional variant thereof can be contacted with the ligand and antibody simultaneously, or sequentially, in either order.

Other methods of identifying the presence of an anti-CD18 antibody can be used, and include numerous antibody-epitope binding assays known in the art. Binding between CD18 and a known or suspected anti-CD18 antibody can alternatively be assessed by assessing a biological function associated with binding between CD18 and a natural ligand thereof (e.g. ICAM-1, ICAM-2, or a cell expressing either of these on its surface) in the presence and absence of the known or suspected anti-CD18 antibody. By way of example, ability of neutrophils to bind with an endothelial cell surface (e.g. the luminal surface of an angioplastied blood vessel) can be assessed in the presence and absence of the known or suspected anti-CD18 antibody, and inhibition of neutrophil binding thereto is an indication that the known or suspected anti-CD18 antibody binds with CD18.

Assays for Chemotaxis or Cellular Stimulation

Chemotaxis assays can be used to assess the ability of an anti-CD18 antibody to block binding of a ligand with a mammalian CD18 or a functional variant thereof. Such assays can also be used to assess the ability of an anti-CD18 antibody to inhibit function associated with binding of the ligand with the mammalian CD18. These assays are based on the functional migration of cells in vitro or in vivo induced by a compound. Chemotaxis can be assessed, e.g. in an assay utilizing a 96-well chemotaxis plate, or using other art-recognized methods for assessing chemotaxis. For example, the use of an in vitro transendothelial chemotaxis assay is described by Springer et al; (Springer et al., WO 94/20142, published Sep. 15, 1994, the teachings of which are incorporated herein by reference; see also Berman et al., Immunol. Invest. 17: 625-677 (1988)). Migration across endothelium into collagen gels has also been described (Kavanaugh et al., J. Immunol., 146: 4149-4156 (1991)). Stable transfectants of mouse L1.2 pre-B cells or of other suitable host cells capable of chemotaxis can be used in chemotaxis assays, for example.

Generally, chemotaxis assays monitor the directional movement or migration of a suitable cell (such as a leukocyte (e.g. lymphocyte, eosinophil, basophil)) into or through a barrier (e.g. endothelium, a filter), toward increased levels of a compound, from a first surface of the barrier toward an opposite second surface. Membranes or filters provide convenient barriers, such that the directional movement or migration of a suitable cell into or through a filter, toward increased levels of a compound, from a first surface of the filter toward an opposite second surface of the filter, is monitored. In some assays, the membrane is coated with a substance to facilitate adhesion, such as ICAM-1, fibronectin, or collagen. Such assays provide an in vitro approximation of leukocyte “homing”.

For example, one can detect or measure inhibition of the migration of cells in a suitable container (a containing means), from a first chamber into or through a microporous membrane into a second chamber which contains an antibody to be tested, and which is divided from the first chamber by the membrane. A suitable membrane, having a suitable pore size for monitoring specific migration in response to compound, including, for example, nitrocellulose, polycarbonate, is selected. For example, pore sizes of about 3-8 microns, and preferably about 5-8 microns can be used. Pore size can be uniform on a filter or within a range of suitable pore sizes.

To assess migration and inhibition of migration, the distance of migration into the filter, the number of cells crossing the filter that remain adherent to the second surface of the filter, the number of cells that accumulate in the second chamber, or both, can be determined using standard techniques (e.g. microscopy). In one embodiment, the cells are labeled with a detectable label (e.g. radioisotope, fluorescent label, antigen or epitope label), and migration is assessed in the presence and absence of the anti-CD18 antibody by determining the presence of the label adherent to the membrane, present in the second chamber, or both, using an appropriate method (e.g. by detecting radioactivity, fluorescence, immunoassay). The extent of migration induced or retarded by an anti-CD18 antibody can be determined relative to a suitable control (e.g. compared to background migration determined in the absence of the antibody, compared to the extent of migration induced by a second compound (i.e. a standard), compared with migration of non-transfected cells induced by the antibody).

In one embodiment, useful for T cells, monocytes, or cells expressing a mammalian CD18, transendothelial migration can be monitored. In this embodiment, transmigration through an endothelial cell layer is assessed. To prepare the cell layer, endothelial cells can be cultured on a microporous filter or membrane, optionally coated with a substance such as collagen, fibronectin, or other extracellular matrix proteins, to facilitate the attachment of endothelial cells. Preferably, endothelial cells are cultured until a confluent monolayer is formed. A variety of mammalian endothelial cells can are available for monolayer formation, including for example, vein, artery or microvascular endothelium, such as human umbilical vein endothelial cells (Clonetics Corp, San Diego, Calif.). To assay chemotaxis in response to a particular mammalian receptor, endothelial cells of the same mammal are preferred; however endothelial cells from a heterologous mammalian species or genus can also be used.

Generally, the assay is performed by detecting the directional migration of cells into or through a membrane or filter, in a direction toward increased levels of a compound, from a first surface of the filter toward an opposite second surface of the filter, wherein the filter contains an endothelial cell layer on a first surface. Directional migration occurs from the area adjacent to the first surface, into or through the membrane, towards a compound situated on the opposite side of the filter. The concentration of compound present in the area adjacent to the second surface, is greater than that in the area adjacent to the first surface.

In one embodiment used to test the effect of an anti-CD18 antibody upon such migration, a composition comprising cells which are capable of migration and which express a mammalian CD18 are placed in the first chamber. A composition comprising one or more ligands or promoters capable of inducing chemotaxis of the cells in the first chamber (having chemoattractant function) is placed in the second chamber. Preferably shortly before the cells are placed in the first chamber, or simultaneously upon placement of the cells, a composition comprising the anti-CD18 antibody to be tested is placed, preferably, in the first chamber. Anti-CD18 antibodies which bind a mammalian CD18-containing protein and inhibit the induction of chemotaxis, by a ligand or promoter, of the cells expressing a mammalian CD18 in this assay are inhibitors of CD18-containing protein-induced function (e.g. inhibitors of stimulatory function). A reduction in the extent of migration induced by the ligand or promoter in the presence of the anti-CD18 antibody is indicative of inhibitory activity. Separate binding studies (see above) could be performed to determine whether inhibition is a result of binding of the antibody to the CD18-containing protein or occurs via a different mechanism.

In vivo assays which monitor leukocyte infiltration into, through, or within a tissue, in response to injection of a compound (e.g. chemokine or antibody) in the tissue, are described below (see Models of Inflammation). These models of in vivo homing measure the ability of cells to respond to a ligand or promoter by emigration and chemotaxis to a site of inflammation and to assess the ability of an anti-CD18 antibody to inhibit or block this emigration.

In addition to the methods described, the effects of an antibody or fragment on the stimulatory function of CD18 can be assessed by monitoring cellular responses induced by active receptor, using suitable host cells containing receptor.

Identification of Additional Ligands, Inhibitors and/or Promoters of Mammalian CD18 Function

The assays described above, which can be used to assess binding and function of the anti-CD18 antibodies described herein, can be adapted to identify additional ligands or other substances which bind a mammalian CD18 or a functional variant thereof, as well as inhibitors and/or promoters of mammalian CD18 function. For example, agents having the same or a similar binding specificity as that of an anti-CD18 antibody (e.g. monoclonal antibody 1B4) can be identified using a competition assay involving the antibody. Thus, the present invention also encompasses methods of identifying ligands or other substances which bind with a mammalian CD18 protein, as well as inhibitors (e.g. antagonists) or promoters (e.g. agonists) of CD18 and CD18-containing protein function. In one embodiment, cells bearing a mammalian CD18 protein or a functional variant thereof (e.g. leukocytes, cell lines or suitable host cells which have been engineered to express a mammalian CD18 protein or a functional variant encoded by a nucleic acid introduced into said cells) are used in an assay to identify and assess the efficacy of ligands or other substances which bind with CD18 or with a CD18-containing protein. Such cells are also useful in assessing the function of the expressed protein or polypeptide (or, of course, of CD18 which is naturally expressed by the cells.

According to the present invention, ligands and other substances which bind with CD18 or a CD18-containing protein, and inhibitors and promoters of a function of such a protein can be identified in a suitable assay, and further assessed for therapeutic effect. Inhibitors of receptor function can be used to inhibit (reduce or prevent) CD18 or a CD18-containing protein activity, and ligands and/or promoters can be used to induce (trigger or enhance) normal CD18 or a CD18-containing protein function where indicated. These inhibitors can be used in methods of treating inflammatory diseases, including autoimmune disease and graft rejection, comprising administering an inhibitor of CD18 or a CD18-containing protein function to an individual (e.g. a mammal). Ligands and/or promoters identified as described herein can be used in a method of stimulating CD18 or a CD18-containing protein function by administering a novel ligand or promoter of protein function to an individual, providing a new approach to selective stimulation of leukocyte function, which is useful, for example, in the treatment of infectious diseases and cancer.

As used herein, a “ligand” of a mammalian CD18 protein refers to a particular class of substances which bind with a mammalian CD18 protein, including natural ligands and synthetic forms, recombinant forms, or both, of natural ligands. Infectious agents having a tropism for mammalian CD18-positive cells can also bind with a mammalian CD18 protein. In a preferred embodiment, ligand binding of a mammalian CD18 protein occurs with high affinity.

As used herein, an “inhibitor” is a substance which inhibits (decreases or prevents) at least one function characteristic of a mammalian CD18 protein (e.g. human CD18; e.g. stimulation of chemotaxis, exocytosis or inflammatory mediator release by leukocytes). The term inhibitor refers to substances including antagonists which bind CD18 or a CD18-containing protein (e.g. an antibody, a mutant of a natural ligand, small molecular weight organic molecules, other competitive inhibitors of ligand binding), and substances which inhibit CD18 or CD18-containing protein function without binding thereto (e.g. an anti-idiotypic antibody).

As used herein, a “promoter” is a substance which promotes (induces, causes, enhances, or increases) at least one function characteristic of a mammalian CD18 protein (e.g. human CD18; e.g. stimulation of chemotaxis, exocytosis or inflammatory mediator release by leukocytes). The term promoter refers to substances including agonists which bind CD18 or a CD18-containing protein (e.g. an antibody, a homolog of a natural ligand from another species), and substances which promote CD18 or CD18-containing protein function without binding thereto (e.g. .by activating an associated protein). In a preferred embodiment, the agonist is other than a homolog of a natural ligand.

Thus, the invention also relates to a method of detecting or identifying an agent which binds a mammalian CD18 protein, including, for example, ligands, inhibitors, promoters, and other substances which bind a mammalian CD18 receptor or a functional variant thereof. According to the method, an agent to be tested, an anti-CD18 antibody described herein (e.g. 1B4, an antibody having an epitopic specificity which is the same as or similar to that of 1B4, and CD18-binding fragments thereof) and a composition comprising a mammalian CD18 protein, a CD18-containing protein, or a ligand binding variant thereof, are combined under conditions suitable for binding of the anti-CD18 antibody to the protein, and binding of the antibody with the protein is assessed, either directly or indirectly, according to methods described herein or other suitable methods. A decrease in the amount of complex formed relative to a suitable control (e.g. in the absence of the agent to be tested) is indicative that the agent binds said receptor or variant. The composition comprising the protein can be a membrane fraction of a cell bearing or comprising the protein. The anti-CD18 antibody can be labeled with a label such as a radioisotope, spin label, antigen or epitope label, enzyme label, fluorescent group and chemiluminescent group.

The assays described above can be used, alone or in combination with each other or other suitable methods, to identify ligands or other substances which bind a mammalian CD18 protein, and inhibitors or promoters of a mammalian CD18 protein or variant. The in vitro methods of the present invention can be adapted for high-throughput screening in which large numbers of samples are processed (e.g. a 96-well format). Cells expressing mammalian CD18 (e.g. human CD18) at levels suitable for high-throughput screening can be used, and thus, are particularly valuable in the identification and/or isolation of ligands or other substances which bind receptor, and inhibitors or promoters of mammalian CD18 proteins. Expression of CD18 or CD18-containing proteins can be monitored in a variety of ways. For instance, expression can be monitored using antibodies of the present invention which bind with such proteins. Also, commercially available antibodies can be used to detect expression of an antigen- or epitope-tagged fusion protein comprising a CD18 (e.g. FLAG tagged receptors), and cells expressing the desired level can be selected.

Nucleic acid encoding a mammalian CD18 protein or a functional variant thereof can be incorporated into an expression system to produce CD18 protein or a CD18-containing protein. Isolated, recombinant, or both, mammalian CD18 protein, CD18-containing protein, or a variant of one of these, such as a protein expressed in cells stably or transiently transfected with a construct comprising a recombinant nucleic acid encoding a mammalian CD18 protein or variant, or in a cell fraction containing receptor (e.g. a membrane fraction from transfected cells, liposomes incorporating the protein), can be used in tests for CD18 function. The protein can be further purified if desired. Testing of CD18 function can be carried out in vitro or in vivo.

Isolated, recombinant, or both, mammalian CD18 protein, CD18-containing protein, or a functional variant of one of these, such as a human CD18, can be used in the present method, in which the effect of a compound is assessed by monitoring receptor function as described herein or using other suitable techniques. For example, stable or transient transfectants (e.g. baculovirus infected Sf9 cells, stable transfectants of mouse L1.2 pre-B cells), can be used in binding assays. Stable transfectants of Jurkat cells or of other suitable cells capable of chemotaxis can be used (e.g. mouse L1.2 pre-B cells) in chemotaxis assays, for example.

According to the method of the present invention, compounds can be individually screened or one or more compounds can be tested simultaneously according to the methods herein. Where a mixture of compounds is tested, the compounds selected by the processes described can be separated (as appropriate) and identified by suitable methods (e.g. PCR, sequencing, chromatography, mass spectroscopy). The presence of one or more compounds (e.g. a ligand, inhibitor, promoter) in a test sample can also be determined according to these methods.

Large combinatorial libraries of compounds (e.g. organic compounds, recombinant or synthetic peptides, “peptoids”, nucleic acids) produced by combinatorial chemical synthesis or other methods can be tested (see e.g. Zuckerman, R. N. et al., J. Med. Chem., 37: 2678-2685 (1994) and references cited therein; see also, Ohlmeyer, M. H. J. et al., Proc. Natl. Acad. Sci. USA 90:10922-10926 (1993) and DeWitt, S. H. et al., Proc. Natl. Acad. Sci. USA 90:6909-6913 (1993), relating to tagged compounds; Rutter, W. J. et al. U.S. Pat. No. 5,010,175; Huebner, V. D. et al., U.S. Pat. No. 5,182,366; and Geysen, H. M., U.S. Pat. No. 4,833,092). Where compounds selected from a combinatorial library by the present method carry unique tags, identification of individual compounds by chromatographic methods is possible.

In one embodiment, a phage display methodology is used. For example, a mammalian CD18 protein, CD18-containing protein, or a functional variant of one of these, an anti-CD18 antibody, and a phage (e.g. a single phage or a plurality or multiplicity of phage, such as a library) displaying a polypeptide, can be combined under conditions appropriate for binding of the antibody with the CD18-containing protein/variant (e.g. in a suitable binding buffer). Phage which can compete with the antibody and bind with the protein can be detected or selected using standard techniques or other suitable methods. Bound phage can be separated from receptor using a suitable elution buffer. For example, a change in the ionic strength or pH can lead to a release of phage. Alternatively, the elution buffer can comprise a release component or components designed to disrupt binding of compounds (e.g. one or more compounds which can disrupt binding of the displayed peptide with the CD18-containing protein/variant, such as a ligand, inhibitor, and/or promoter which competitively inhibits binding). Optionally, the selection process can be repeated or another selection step can be used to further enrich for phage which bind with the protein/variant. The displayed polypeptide can be characterized (e.g. by sequencing phage DNA). The polypeptides identified can be produced and further tested for binding, and for inhibitor or promoter function. Analogs of such peptides can be produced which will have increased stability or other desirable properties.

In one embodiment, phage expressing and displaying fusion proteins comprising a coat protein with an amino-terminal peptide encoded by random sequence nucleic acids can be produced. Suitable host cells expressing a CD18-containing protein/variant and an anti-CD18 antibody are combined with the phage, bound phage are selected, recovered and characterized. (See. e.g. Doorbar and Winter, J. Mol. Biol. 244:361 (1994), discussing a phage display procedure used with a G protein-coupled receptor, and WO 97/08320 (Morphosys), published Mar. 6, 1997).

Other sources of potential ligands or other substances which bind to, or inhibitors, promoters, or both, of mammalian CD18 proteins include, but are not limited to, variants of known CD18 ligands, including naturally occurring, synthetic, or recombinant variants of ICAM-1, ICAM-2, ICAM-3, C3bi, factor X, fibrin, and fibrinogen, substances such as other chemoattractants or chemokines, variants thereof, low molecular weight organic molecules, other inhibitors and/or promoters (e.g. anti-CD18 antibodies, antagonists, agonists), inhibitors and/or promoters (e.g. antagonists or agonists), and soluble portions of a mammalian CD18, such as a suitable receptor peptide or analog which can inhibit CD18 function.

Models of Inflammation

In vivo models of inflammation are available which can be used to assess the effects of anti-CD18 antibodies in vivo as therapeutic agents. For example, leukocyte infiltration upon intradermal injection of a chemokine and an antibody or fragment thereof reactive with mammalian CD18 into a suitable animal, such as rabbit, mouse, rat, guinea pig or rhesus macaque (preferably a primate, such as a Cynomolgus monkey) can be monitored (see e.g. Van Damme, J. etal., J. Exp. Med., 176: 59-65 (1992); Zachariae, C.O.C. etal., J Exp. Med. 171: 2177-2182 (1990); Jose, P.J. et al., J Exp. Med. 179: 881-887 (1994)). In one embodiment, skin biopsies are assessed histologically for infiltration of leukocytes (e.g. eosinophils, granulocytes). In another embodiment, labeled cells (e.g. stably transfected cells expressing a mammalian CD18, labeled with ¹¹¹In for example) capable of chemotaxis and extravasation are administered to the animal. For example, an anti-CD18 antibody to be assessed can be administered, either before, simultaneously with or after ligand or agonist is administered to the test animal. A decrease of the extent of infiltration in the presence of antibody as compared with the extent of infiltration in the absence of inhibitor is indicative of inhibition.

Diagnostic, Therapeutic, and Preventive Methods

It has been discovered that molecules which bind specifically with the CD18 subunit of the Mac-1 leukocyte cell-surface antigen (and other CD18-containing proteins such as LFA-1, p150,95, and CD11d/CD18) inhibit interactions of leukocytes with vascular endothelium. Interactions which are inhibited by such molecules include

binding of leukocytes with vascular endothelium,

translocation of leukocytes through vascular endothelium,

infiltration of leukocytes into intimal vascular tissue,

release of a chemotactic factor from leukocytes in vascular tissue,

release of a growth factor from leukocytes in vascular tissue,

leukocyte-binding-associated release of a chemotactic factor from vascular tissue, and

leukocyte-binding-associated release of a growth factor from vascular tissue.

Thus, molecules which bind specifically with CD18 so as to inhibit interaction of a CD18-containing protein (especially including a CD18-containing leukocyte cell-surface antigen) can be used to inhibit, prevent, or reverse any of these leukocyte-vascular endothelium interactions.

The invention thus includes a method of inhibiting, or even preventing, stenosis (including restenosis) in a mammalian blood vessel. This method comprises administering to the mammal an anti-CD18 antibody which binds specifically with at least the CD18 portion of a mammalian protein which comprises CD18. Stenosis is thereby inhibited in the vessel in the mammal. Substantially the same method can be used to alleviate existing stenosis and associated disorders in mammalian blood vessels. For example, this method can be used to inhibit or prevent stenosis or restenosis in human coronary and cerebral blood vessels or to alleviate existing stenosis in such vessels. Symptoms and disorders associated with such stenoses can likewise be inhibited, prevented, or alleviated. Symptoms associated with stenosis and restenosis include, for example, short- and long-term ischemia in tissues located upstream and downstream from the stenosed site and attendant pain and tissue necrosis. Examples of disorders associated with stenoses include both disorders which can be brought about by existence of the stenosis (e.g. myocardial infarction) and disorders in which the stenosis is simply a symptom of the disorder (e.g. hypercholesterolemia and associated atherosclerosis.

Stenoses which can be inhibited, prevented, or alleviated using the methods described herein include stenoses caused by surgical, angioplastic, or other intentional intervention which perturbs the vascular endothelium. Examples of such interventions include, for example, angiography, angioplasty (e.g., performed by balloon, atherectomy, laser angioplasty or other suitable methods, with or without each of rotablation and stent placement), endarterectomy, coronary artery by-pass surgery, stent placement (e.g., endovascular stent, coronary stent), other vascular intervention procedures (e.g., vascular surgery, vascular graft, deployment of a peripheral stent), insertion of a prosthetic valve or vessel (e.g., an autologous, non-autologous or synthetic vessel graft), transplantation of an organ, tissue, or cell, and intravascular brachytherapy. In a particular aspect, the method can be used to inhibit, prevent, or alleviate stenosis or restenosis which occurs following a coronary artery intervention procedure, such as percutaneous transluminal coronary angioplasty (PTCA), or a vascular intervention procedure which includes placement of a stent (e.g., PTCA followed by placement of an endovascular stent with one or more coronary arteries).

Types of vascular endothelial perturbations that can lead to stenosis which can be inhibited, prevented, or alleviated using the methods described herein include, for example, endothelial denudation of blood vessels, dissection of a layer of vascular tissue (e.g. endothelium, intimal tissue, elastic layer, etc.) from another layer of vascular tissue, rupture of the tunica intima tissue layer of a blood vessel, and intentional incision of vascular tissue layers (e.g. using a cutting angioplasty balloon).

Vascular injury can be diagnosed in patients using any of a variety of known methods. Examples of such methods include X-ray fluoroscopic exarnination of dye flowing through a particular region of a blood vessel, the presence of symptoms such as pain, based on clinical judgment, or signs evidenced on physical examination. Alternatively, it can be assumed that injury will arise upon performance of one of the surgical or other intentional vascular interventions discussed above, or following diagnosis of a disease or disorder known to be associated with vascular perturbation (e.g. atherosclerosis) in a patient.

The anti-CD18 antibody or antibodies which can be used in these stenosis-inhibiting and -alleviating methods can be substantially any of the anti-CD18 antibodies described herein. For example, the antibody can be monoclonal antibody 1B4 or an antibody which exhibits the same epitopic specificity as 1B4 (i.e. on which binds specifically with the same epitope of CD18 with which 1B4 binds). Thus, in one embodiment, the antibody which is used binds specifically with substantially only the CD18 portion of the CD18-containing protein (e.g. with substantially only the CD18 subunit of the Mac-1 leukocyte cell-surface antigen).

While not being bound by any particular theory of operation, it is believed that binding of the anti-CD18 antibody with the CD18-containing protein inhibits binding of a natural ligand of the protein with that protein (e.g. binding of Mac-1 with ICAM-1 can be inhibited). Inhibition of binding between the CD18-containing protein and a natural ligand thereof inhibits the physiological effect (e.g. leukocyte attachment to vascular endothelium) which is normally associated with such binding. When the physiological effect is one which is associated with stenosis, the stenosis is inhibited, prevented, or alleviated by inhibiting the physiological effect. Thus, it is believed that the inhibitory/preventive/palliative effect of anti-CD18 antibodies on stenoses and their associated symptoms and disorders is attributable to inhibition of interactions between leukocytes and cells of endothelial and intimal vascular tissues mediated by binding of cell-surface CD18-containing proteins of the leukocytes and receptor molecules on the surface of the vascular cells, in the extracellular matrix surrounding the vascular cells, or both.

It is believed that inhibition by anti-CD18 antibodies of interactions between neutrophils and vascular cells (particularly including vascular endothelial cells) accounts for a significant degree of the anti-stenotic effects attributable to administration of such antibodies. Nonetheless, it is recognized that CD18-containing proteins occur on the cell surfaces of other leukocytes, including lymphocytes, monocytes, granulocytes, T cells, and basophils. By binding with CD18-containing proteins, anti-CD18 antibodies can inhibit the contribution of these cells to symptoms and disorders associated with stenosis, and can furthermore inhibit the contribution of these cells to the stenosis itself.

Although the operability of the methods described herein does not depend on the theories set forth to explain that operability, the following are believed to be true. Integrins are a recognized class of leukocyte cell surface proteins which contain CD 18. Each of the integrins is a heterodimer, consisting of a CD11 isotype (i.e. one of CD11a, CD11b, CD11c, and CD11d) monomer and a CD18 monomer. Integrins bind specifically with cell-surface proteins of other cells and with proteins of the extracellular matrix. Proteins with which integrins are known to bind specifically include, for example, ICAM-1, ICAM-2, ICAM-3, C3bi, factor X, fibrin, and fibrinogen. The Mac-1 antigen (CD11b/CD18), for example, is known to bind specifically with ICAM-1, C3bi, factor X, fibrin, and fibrinogen. Binding of an anti-CD18 antibody with an integrin can inhibit binding between the integrin and one or more of these factors, some of which occur in vascular extracellular matrix and on the surface of vascular cells (including vascular endothelial cells). To the extent that binding of an integrin with one or more of these ligands facilitates binding of leukocytes with vascular tissue, infiltration of leukocytes through or into vascular tissue, release of chemotactic (chemoattractant or chemorepellant) factors or growth factors from either leukocytes or vascular cells, or any other symptom associated with stenosis, inhibition of such binding alleviates, inhibits, or even reverses such symptoms.

The anti-CD18 antibodies described herein can be used to treat stenosis in substantially any mammalian blood vessel in which they occur, including in blood vessels in which restenosis recurs or threatens to recur following treatment of stenosis (e.g. in a coronary artery following percutaneous transluminal coronary angioplasty using an ordinary or cutting balloon angioplasty device). The mammalian blood vessel is preferably a blood vessel of a human, particularly when a human or humanized antibody (e.g. monoclonal antibody 1B4) is used.

Blood vessels in which stenosis (and restenosis) commonly occurs include those in which the vascular endothelium has been traumatically perturbed, such as by a surgical or angioplastic intervention or by a traumatic injury (including injuries of a ‘crushing’ sort). Examples of blood vessels having traumatically perturbed endothelium include blood vessels which are grafted to one another (i.e. including both the implanted vessel segment and the segments with which it is grafted, regardless of the identity or identities of the source of the implanted segment and the host), blood vessels which have been surgically punctured or slit, blood vessels within which an angioplasty balloon or other expandable member has been expanded, blood vessels comprising a portion at which a laser angioplasty procedure has been performed in order to ablate a portion of the vessel or a plaque or other obstruction therein, blood vessels with which a stent has been emplaced, and the like.

Stenosis is also known to occur in blood vessels which have not experienced traumatic injury to the endothelium, but in which gradual deterioration or degradation of the endothelium has occurred, often accompanied by intimal thickening, deposition of cells, lipid, minerals, or other extracellular material, or both. Examples of disorders which can lead to gradual degradation of vascular endothelium include atherosclerosis and arteriosclerosis. Because atherosclerosis is believed to be attributable, at least in part to localization of leukocytes at a site of vascular endothelial injury, subsequent plaque formation, and eventual stenosis, the preventive and therapeutic methods described herein can be used to inhibit or prevent atherosclerosis before stenosis occurs, and even at very early stages of the disorder. Furthermore, those therapeutic methods can be used to alleviate existing atherosclerosis by, for example, inducing shrinkage or disappearance of atherosclerotic plaques and by inhibiting localization of leukocytes at sites of vascular endothelial injury. Thus, “inhibition, prevention, or alleviation” of stenosis includes inhibition, prevention, or alleviation of atherosclerosis, even at relatively early (i.e. long before imminent occurrence of stenosis) stages of this disorder.

The therapeutic and preventive methods described herein can be used to inhibit, prevent, or alleviate stenosis of substantially any blood vessel in which stenoses (or restenoses) occur, it being understood that the methods are not necessarily applicable to congenital stenosis, such as aortic root and aortic valve stenoses, which appear to be unrelated to interaction of leukocytes with vascular tissues. For example, the methods described herein can be used to treat, prevent, or inhibit stenoses of coronary and cerebral blood vessels.

The anti-CD18 antibody used in the therapeutic and preventive methods described herein can be any of the types of anti-CD18 antibodies that are described in this disclosure. For example, a whole antibody can be used (e.g. an isolated murine antibody which specifically binds with human CD18). Alternatively, the anti-CD18 antibody can be a fragment of a whole antibody, such as a Fv, Fab, Fab′, or F(abN)₂ fragment. The anti-CD18 antibody can be an antibody isolated from a human, from a non-human mammal, from a non-human vertebrate, from a library of random or synthetic antibodies. Furthermore, the anti-CD18 antibody can be an antibody which comprises segments obtained from different sources (i.e. a chimeric antibody). By way of example, the antibody can have complementarity-determining regions which have the amino acid sequence of the same regions of a murine antibody which binds specifically with CD18; the same antibody can have non-complementarity-determining regions (also designated structural, framework, or constant regions) which have amino acid sequences which are derived from one or more human antibodies or from consensus human antibody sequences. This antibody is also an example of a type of humanized antibody (i.e. an antibody in which at least a part of the antibody is derived from a non-human source, but which has been modified such that at least one other part of the antibody is more nearly like a human antibody in terms of its amino acid sequence). Anti-CD18 antibodies which are humanized, using any of the methods described herein or any other method known in the art or hereafter developed, can be used in any of the methods described in this disclosure.

The route and method used to administer the anti-CD18 antibody to the mammal according to the methods described herein is not critical. All that is necessary is that the anti-CD18 antibody is provided, in a non-denatured form, to the cell(s) or tissue(s) of the blood vessel at which it is to act or to leukocytes which can contact the blood vessel cell(s) or tissue(s). That is, the anti-CD18 antibody can be provided to a vascular tissue (e.g. vascular intimal tissue or vascular endothelial tissue) directly, such as by injection into the tissue or by topical application to the tissue after surgically exposing the tissue. Alternatively, and preferably, the anti-CD18 antibody can be administered to the mammal by providing the anti-CD18 antibody to the blood stream, whence the antibody can bind with CD18-containing proteins on the surface of leukocytes present in the blood stream. Administration of the anti-CD18 antibody to lymph, to the spleen, to the thymus, or to other sites at which leukocytes can be found in the mammal can also be used.

Where possible, administration of the anti-CD18 antibody to the bloodstream of the mammal preferably occurs prior to disruption of the vascular endothelium (e.g. prior to surgical or angioplastic intervention in a blood vessel of the mammal). Nonetheless, the anti-CD18 antibody can be administered to the mammal at the time of vascular endothelial disruption or even significantly thereafter, particularly when the stenotic disorder which is being alleviated is an on-going disorder or one which can go undetected for a significant period prior to treatment (e.g. atherosclerosis). Thus, the anti-CD18 antibody can be administered before, during, or following injury to vascular tissue.

It is advantageous to maintain the anti-CD18 antibody concentration in the mammal's bloodstream at a level which is sufficient to maintain substantially all, or at least a large fraction (i.e. 50%, 60%, 70%, 80%, 90%, or 95% or more), of the CD18-containing leukocyte cell-surface antigens occupied with at least one copy of the antibody. This can be achieved by administering multiple doses of the anti-CD18 antibody or by administering a sustained-release composition of the antibody to the mammal. The antibody content and timing of doses can be determined experimentally by routine experimentation. It is understood that the half-life (i.e. the functional life span) of the anti-CD18 antibody will be greater the more the anti-CD18 antibody resembles an antibody which is normally produced by the mammal to which it is administered. Thus, when the mammal is a human, it is preferable to use a human or humanized antibody, in order to reduce the amount of anti-CD18 antibody which needs to be administered, as well as the frequency with which it need be administered.

Typically, an effective amount can range from about 0.01 milligram per day to about 100 milligrams per day for an adult. Preferably, the dosage ranges from about 1 milligram per day to about 100 milligrams per day. Antibodies and antigen-binding fragments thereof, particularly human, humanized and chimeric antibodies and antigen-binding fragments can often be administered with less frequency than other types of therapeutics. For example an effective amount of an antibody can range from about 0.01 milligrams per kilogram body weight to about 5 or 10 milligrams per kilogram body weight administered daily, weekly, biweekly, or monthly.

One or more of the anti-CD18 antibodies described herein can be packaged in the form of a kit containing the antibody(ies) and an instructional material which describes administration of one or more of the antibodies for inhibition, prevention, or alleviation of stenosis. The instructional material can further include, for example, a description of relevant dosing and administration information for a pharmaceutical composition comprising the anti-CD18 antibody(ies).

The anti-CD18 antibodies described herein can be used to detect cells and tissues which bear (i.e. exhibit on their surface or at an extracellular solvent-accessible location) a CD18-containing protein. The anti-CD18 antibodies can also be usedto detect CD18-containing protein in a sample (e.g. a cell homogenate) in which cellular and tissue structures have artificially been rearranged from their naturally-occurring orientations. Detection of CD18-containing proteins in a cell, tissue, or body fluid (e.g. blood) sample can indicate the presence of cells (e.g. leukocytes) which bear such proteins. The anti-CD18 antibody can be labeled with, for example, a fluorescent, chromatic, enzymatic, or radioactive label (e.g. a gamma radiation source such as a gamma radiation-emitting radionuclide) in order to simplify detection of the antibody (and protein, cells, or tissues with which it may be bound).

The presence of cells which bear CD18-containing proteins, particularly at a level that is greater than normal for the individual from whom the sample was taken or at a level greater than the level or range of levels that naturally occurs in a patient not afflicted with, developing, or pre-disposed to develop a stenosis or stenosis-related disorder, can indicate that the subject from whom the sample was obtained is pre-disposed to develop, is developing, or is presently afflicted with, such a disorder. For convenience of the medical practitioner, the components of this kit.(i.e. one or more anti-CD18 antibodies and an instructional material) can be packaged in the form of a kit for assessing the presence of CD18-containing proteins in a sample obtained from a patient.

The anti-CD18 antibodies described herein are useful in a variety of applications, including research applications. In one embodiment, the antibodies are labeled with a suitable label (e.g. fluorescent label, chemiluminescent label, isotope label, antigen or epitope label or enzyme label). For instance, they can be used to isolate and/or purify CD18 protein, CD18-containing proteins, natural variants thereof, or portions thereof, and to study CD18 structure (e.g. conformation) and function.

In addition, the various antibodies of the present invention can be used to detect CD18 or to measure the expression of the protein, for example, on T cells (e.g. CD26+ cells, CD45RO+ cells), neutrophils, eosinophils, or on cells transfected with a receptor gene. Thus, they also have utility in applications such as cell sorting (e.g. flow cytometry, fluorescence activated cell sorting), for diagnostic or research purposes.

Typically, diagnostic assays entail detecting the formation of a complex resulting from the binding of an anti-CD18 antibody with CD18 or with a CD18-containing protein. For diagnostic purposes, the antibody(ies) can be labeled or non-labeled. The antibody can, for example, be directly labeled. A variety of labels can be employed, including, but not limited to, radionuclides, fluorophores, enzymes, enzyme substrates, enzyme cofactors, enzyme inhibitors and ligands (e.g. biotin, haptens). Numerous appropriate immunoassays are known to the skilled artisan (see, for example, U.S. Pat. Nos. 3,817,827; 3,850,752; 3,901,654 and 4,098,876). When unlabeled, the anti-CD18 antibody can be detected using suitable means, as in agglutination assays, for example. Non-labeled anti-CD18 antibodies can also be used in combination with one or more suitable reagents which can be used to detect the antibody, such as a labeled antibody (e.g. a second antibody) which binds specifically with the anti-CD18 antibody (e.g. anti-idiotype antibodies or other antibodies that are specific for the non-labeled immunoglobulin) or other suitable reagent (e.g. labeled protein A).

In one embodiment, the anti-CD18 antibodies described herein can be used in enzyme immunoassays, wherein the subject antibody, or an antibody which binds specifically therewith, is conjugated with an enzyme. When a biological sample comprising a mammalian CD18 protein is combined with the antibody(ies), binding occurs between the anti-CD18 antibody and CD18 protein. In one embodiment, a sample containing cells expressing a mammalian CD18 protein, such as human blood, is combined with the antibodies, and binding occurs between the antibodies and cells bearing a human CD18 protein (i.e. cells comprising an epitope recognized by the anti-CD18 antibody). These bound cells can be separated from non-bound reagents, and the presence of the antibody-bound cells can be determined, for example, by contacting the sample with a substrate of the enzyme which produces a color or other detectable change when acted on by the enzyme. In another embodiment, the subject antibodies can be unlabeled, and a second, labeled antibody can be added which recognizes the subject antibody.

Kits useful for detecting the presence of a mammalian CD18 protein in a biological sample can also be prepared in view of the present disclosure. Such kits include an anti-CD18 antibody and one or more ancillary reagents suitable for detecting the presence of a complex between the antibody and CD18. The antibody can be provided in a lyophilized form, either alone or in combination with additional antibodies which bind specifically with other epitopes. The antibodies, which can be labeled or non-labeled, can be included in the kits with adjunct ingredients (e.g. buffers, such as Tris, phosphate, and carbonate buffers, stabilizers, excipients, biocides, inert proteins {e.g. bovine serum albumin}, or some combination of these). For example, the antibodies can be provided as a lyophilized mixture with the adjunct ingredients, or the adjunct ingredients can be separately provided for combination by the user. Generally these adjunct materials are present in less than about 5% weight based on the amount of active antibody, and can be present in a total amount of at least about 0.001% weight based on antibody concentration. When a second antibody is included in the kit, the second antibody being capable of binding specifically with the anti-CD18 antibody, the second antibody can be provided in the kit, for instance in a separate vial or container. The second antibody, if present, can be labeled, and can be formulated in an analogous manner with the antibody formulations described above.

Similarly, the present invention relates to a method of detecting and/or quantitating expression of a mammalian CD18 or a portion of CD18 by a cell. According to this method, a composition comprising the cell or fraction thereof (e.g. a membrane fraction) is contacted with an anti-CD18 antibody (e.g. monoclonal antibody 1B4) under conditions appropriate for binding of the antibody with the protein, and binding is monitored. Detection of the antibody, indicative of the formation of a complex between antibody and CD18, indicates the presence of the protein. Binding of antibody with the cell can be determined as described above under the heading “Binding Assays”, for example. The method can be used to detect expression of CD18 on cells obtained from an individual (e.g. in a sample, such as blood, saliva or other body fluid). The level of expression of CD18 on the surface of T cells or monocytes can also be determined, for instance, by flow cytometry, and the level of expression (e.g. staining intensity) can be correlated with disease susceptibility, progression or risk.

Another use of the anti-CD18 antibodies described herein is for removing leukocytes which bind specifically with an anti-CD18 antibody from a mammal's blood. By removing (or at least reducing the number of) such cells from the subject's blood stream, stenosis and associated symptoms and disorders can be inhibited, prevented, or alleviated in a blood vessel of the subject. By way of example, anti-CD18 antibodies can be fixed to a solid support, and blood obtained from the subject can be contacted with the support (whereupon cells bearing CD18-containing proteins bind with the support) prior to returning the blood to the subject's blood stream.

Modes of Administration

One or more anti-CD18 antibodies described herein can be administered to an individual by an appropriate route, either alone or in combination with (before, simultaneous with, or after) another drug or agent. For example, the antibodies of the present invention can be used in combination with other monoclonal or polyclonal antibodies (e.g. in combination with antibodies which bind other leukocyte cell-surface antigens, including, but not limited to, cell-surface immunoglobulin receptors and selectins) or with existing blood plasma products, such as commercially available gamma globulin and immune globulin products used in prophylactic or therapeutic treatments. The anti-CD18 antibodies can be used as separately administered compositions given in conjunction with antibiotics and/or anti-microbial agents. The anti-CD18 antibodies can also be administered in combination with anti-viral agents, immunosuppressive agents (e.g. calcineurin inhibitors, such as cyclosporin A; glucocorticoids, such as prednisone or methylprednisolone; and nucleic acid synthesis inhibitors, such as azathioprine or mycophenolic acid), cytokines, such as interferon and Th2-producing cytokines, and hormones, such as adrenocortictropic hormone.

An effective amount of an antibody or fragment (i.e. one or more antibodies or fragments) is administered. An effective amount is an amount sufficient to achieve the desired therapeutic (including prophylactic) effect, under the conditions of administration, such as an amount sufficient for inhibition of a CD18 function, and thereby, inhibition, prevention, or alleviation of a stenosis, or a symptom or disorder associated with a stenosis, in a human.

A variety of routes of administration are possible including, but not necessarily limited to, oral, dietary, topical, parenteral (e.g. intravenous, intraarterial, intramuscular, subcutaneous injection), inhalation (e.g. intrabronchial, intraocular, intranasal or oral inhalation, intranasal drops), intraocular, depending on the disease or condition to be treated. Other suitable methods of administration can also include rechargeable or biodegradable devices and slow release polymeric devices. The pharmaceutical compositions of this invention can also be administered as part of a combinatorial therapy with other agents.

Formulation of an anti-CD18 antibody to be administered will vary according to the route of administration and formulation (e.g. solution, emulsion, capsule) selected. An appropriate pharmaceutical composition comprising an anti-CD18 antibody to be administered can be prepared in a physiologically acceptable vehicle or carrier. A mixture of antibodies can also be used. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. A variety of appropriate aqueous carriers are known to the skilled artisan, including water, buffered water, buffered saline, polyols (e.g. glycerol, propylene glycol, liquid polyethylene glycol), dextrose solution and glycine. Intravenous vehicles can include various additives, preservatives, or fluid, nutrient or electrolyte replenishers (See, generally, Remington's Pharmaceutical Science, 16th Edition, Mack, Ed. 1980). The compositions can optionally contain pharmaceutically acceptable auxiliary substances as required to approximate physiological conditions such as pH adjusting and buffering agents and toxicity adjusting agents, for example, sodium acetate, sodium chloride, potassium chloride, calcium chloride and sodium lactate. The anti-CD18 antibodies fragments of this invention can be lyophilized for storage and reconstituted in a suitable carrier prior to use according to art-known lyophilization and reconstitution techniques. The optimum concentration of the active ingredient(s) in the chosen medium can be determined empirically, according to procedures well known to the skilled artisan, and will depend on the. ultimate pharmaceutical formulation desired. For inhalation, the antibody or fragment can be solubilized and loaded into a suitable dispenser for administration (e.g. an atomizer, nebulizer or pressurized aerosol dispenser).

The articles “a” and “an” are used herein to refer to one or to more than one (i.e. to at least one) of the grammatical object of the article. By way of example, “an element” means one element or more than one element.

The invention is now described with reference to the following Example. This Example is provided for the purpose of illustration only, and the invention is not limited to this Example, but rather encompasses all variations which are evident as a result of the teaching provided herein.

### EXAMPLE

In the experiments presented in this example, use of murine monoclonal anti-CD18 antibodies which specifically bind with human CD18 in a Cynomolgus monkey model of restenosis was evaluated.

Cynomolgus monkeys were randomized on the basis of body weight to groups to receive treatment with either an irrelevant murine monoclonal antibody (mAb) as an IgG2a isotype control (S-S.1) or an anti-human CD18 mAb (1B4). Animals were administered a loading dose of mAb intravenously (IV) on Day −1, followed by daily SC injections on Days 1-13. On Day 1, all animals underwent bilateral balloon angioplasty-induced iliac artery endothelial denudation, followed by intravascular stent placement, as a model of restenosis. Animals were euthanized at the end of the test period to allow perfusion fixation and collection of the iliac arteries and other tissue samples (see Table A).

Efficacy of treatment was evaluated by use of quantitative angiography at the time of stent placement and at the end of the study, and by immunohistologic and morphometric evaluation of iliac artery tissue. Blood samples were collected periodically for assay of serum mAb levels (pharmacokinetics), leukocyte mAb binding (pharmacodynamics), anti-mAb antiglobulin response (immunogenicity), and for hematology and serum chemistry (safety). Safety was further evaluated by recording vital signs during infusion and body weights, clinical observations and injection site observations during the test period. Other tissue samples were not be evaluated unless warranted (see Table B).

TABLE A
Study Design

| | | Treatment | | | |
|---|---|---|---|---|---|
| Group No. | No. | Test | Dose and Dose | | |
| (Description) | Animals | Materials | Regimen | Model of Restenosis | Euthanasia |
| 1 | 5 | S-S.1 | 5 mg/kg, IV, | Bilateral balloon | Day 29 |
| (IgG2a | | | in 30 mL | angioplasty-induced iliac | |
| control) | | | over 30 min | artery endothelial | |
| | | | on Day −1; | denudation and | |
| | | | | intravascular stent | |
| | | | | placement on Day 1 | |
| 3 | 5 | 1B4 | 1 mg/kg, SC, | | |
| (anti-CD18) | | | in 3 mL | | |
| | | | on Days 1-13 | | |

There is no Day 0; Day −1 precedes Day 1. Day 1 was not the same calendar day for all animals.
Day −1 treatment was via peripheral vein. Day 1-13 SC treatment was given in the intrascapular area. Day 1 treatment was prior to angioplasty/stenting.
Doses were based on Day −1 body weight and were maintained throughout the treatment period.
IV = intravascular;
SC = subcutaneous;
M = male.

TABLE B
Study Procedures

| | PK | | IMG | | | | | | | | |
|---|---|---|---|---|---|---|---|---|---|---|---|
| | Sera mAb | PD | Anti-mAb | Safety | | | | | | | Efficacy |
| Study | conc. | mAb binding | Abs | Hem | SC | | Clinical | Injection | Vital | | Quantitative |
| Day | Blood Collection | | | | | BW | Obs. | Site Obs. | Signs | Nx | Angiography |
| BL | X | X | X | X | X | X | | | | | |
| −1 | X(pre) | X(pre) | X(pre) | X(pre) | X(pre) | X | Day −1(pre | | X | | |
| | X(post) | X(post) | | | | | and post) | | | | |
| 1 | X | X | X | | | | then daily | Day 1(pre | | | X |
| 8 | X | X | X | X | X | X | | and post) | | | |
| 15 | X | X | X | X | X | X | | then daily | | | |
| 22 | X | X | X | X | X | X | | | | | |
| 29 | X | X | X | X | X | X | | | | X | X |
| Vol. | 1.0 | 1.0 | 1.0 | 0.5 | 1.0 | | | | | | |
| (mL) | | | | | | | | | | | |
| Anticoagulant | None | Heparin | None | EDTA | None | | | | | | |
| Notes | a (3^{rd}) | b (1^{st}) | a (3^{rd}) | c (2^{nd}) | d (4^{th}) | | | | | | |

a = Prioritize as 3^{rd} sample(s). Freeze (−70° C.) in 100 μL aliquots.
b = Prioritize as 1^{st} sample.
c = Prioritize as 2^{nd} sample collected. Use microtainers.
d = Prioritize as 4^{th} sample. Freeze (−70° C.) residual sera in single aliquot.
Procedures (except vital signs, angiography and Nx) were done prior to treatment, unless otherwise specified.
Abbreviations:
Abs = antibodies;
BL = baseline;
BW = body weight;
Hem = hematology;
IMG = immunogenicity;
mAb = monoclonal antibody;
Nx = euthanasia, perfusion and tissue collection;
PD = pharmacodynamics;
PK = pharmacokinetics;
pre/post = pre- and post-infusion;
SC = serum chemistry;
X = was performed.

Disease Model

Atherosclerosis is a disease in humans in which lipid-rich fibro-inflammatory plaques accumulate within the wall of the coronary vessels, encroaching upon and narrowing (“stenosing”) the lumen, thus limiting oxygenated blood supply to cardiac tissue and resulting in acute myocardial pain and/or infarction. Current medical practice to address compromised coronary vessels involves mechanical dilatation of the vessel with a balloon catheter via percutaneous transluminal coronary angioplasty (PCTA), often followed by placement of an intravascular stent to maintain luminal diameter (1). In a significant number of patients, late(r) restenosis limits the effectiveness of this procedure (2). Neointimal hyperplasia, vascular smooth muscle cell (VSMC) proliferation and infiltrative leukocytes characterize the area of restenosis. Possible mechanisms involved in this process include platelet aggregation (thrombosis), endothelial cell activation and VSMC proliferation and migration. A variety of animal models of atherosclerosis and/or restenosis have been developed, in species such as mice, rats, rabbits, pigs, and non-human primates (Cynomolgus monkeys and baboons). The model of neointimal hyperplasia used in this study, balloon angioplasty-induced endothelial denudation followed by stent placement, has been previously used in rabbits to elucidate some of the mechanisms involved in restenosis (4).

Test Materials

1B4 is a murine IgG2a mAb that recognizes CD18 on human, non-human primate and rabbit neutrophils. 1B4 was produced using a commercially available cell line that makes the antibody (ATCC Accession No. TIB10164). S-S.1 is a murine IgG2a mAb directed against sheep red blood cells. S-S.1 was produced using a commercially available cell line that makes the antibody (ATCC Accession No. TIB-111) and is being used as an irrelevant isotype-matched control antibody.

Dose and Dose Regimen

The dose and dose regimen were selected because they are anticipated to result in peak and trough sera mAb concentrations in excess of those required to maintain continuous saturation of CD18 on leukocytes through at least Day 14. It is recognized that neutralizing monkey anti-mouse mAb antiglobulin (MAMA) responses will develop in these animals and that these responses may affect sera or cell-bound mAb levels and thus PK, PD and/or efficacy endpoints.

Vital Sign Monitoring

These mAbs, as with many other antibodies, have the potential to induce a “first-dose effect” related to cytokine release during initial infusion, or to precipitate ADCC (antibody-dependent cell-mediated cytotoxicity) or complement-mediated cell lysis. These effects can result in transient adverse physiologic changes, such as hypotension and bronchoconstriction, which are usually not life threatening. Monitoring vital signs allows detection of such changes.

Test System

The murine anti-human CD18 mAb also binds Cynomolgus monkey CD18.

Number of Animals

The number of animals used in this study was sufficient for evaluation of the results. Although 4 animals/group has previously been sufficient to allow detection of efficacy in a rabbit model (4), it was considered appropriate to use 5 animals/group in this study because of potentially greater variability in degree of vascular injury and response thereto in monkeys.

The test materials and formulations used in the experiments presented in this example are now described.

Characterization

The mAb solutions were biochemically characterized prior to use (see Table C).

Stability

Samples of the test articles were retrieved from the test site at completion of dosing and characterized biochemically. No significant changes in the samples, relative to the original characterization, were detected.

Dose Formulation Methods

On the day(s) of use, an appropriate number of vials of the frozen mAb solutions were brought to room temperature and appropriate volumes diluted in vehicle (saline) as necessary to provide uniform total volumes for IV (30 mL in a 60 cc syringe) or SC (3 mL in a 3 cc syringe) administration to all animals. The date of thawing was recorded on the vial(s). Unused (thawed, opened) bulk mAb solutions were refrigerated (2-8° C.) for use on subsequent day(s).

Dose Formulation Samples

No dose formulation samples were collected.

Disposition

Residual diluted dose formulations were discarded.

TABLE C
Test Materials

| | | Storage | Physical | | | | |
|---|---|---|---|---|---|---|---|
| Identification | Conc. | Conditions | Description | Supplier | Manufacturer | Lot No. | Biohazards |
| Saline | N/A | Ambient | Clear liquid | Primedica | TBD | TBD | None; |
| (Vehicle for | | | | | | | Use standard |
| dilution) | | | | | | | precautions |
| S-S.1 | 4.2 mg/mL | −70° C. | Clear to | LeukoSite | LeukoSite | TBD | |
| (Irrelevant | | until | slightly | | | | |
| IgG2a control | | thawed, | cloudy | | | | |
| mAb)^{a} | | then | solution | | | | |
| 1B4 | 5 mg/mL | 2-8° C. | | | LeukoSite | TBD | |
| (Anti-CD18 | | | | | | | |
| mAb)^{b} | | | | | | | |

^{a}Cell line obtained from ATCC; No. TIB-111 also referred to S-S.1.
^{b}Cell line obtained from ATCC; No. HB-10164.
TBD = documented in study file;
ATCC: American Type Culture Collection

The test system used in the experiments presented in this example is now described.

Animals

Species: Macaca fascicularis

Common name: Cynomolgus monkey

Number of Animals: 15

Age and Gender: Young-adult males

Weight at Initiation of Treatment: ˜4 kg

Source and Selection

Animals were obtained from a source approved by the Testing Facility. Animals were be selected from those available at the time of the study and appeared to be in good health, as determined by a veterinarian. All animals completed a period of quarantine, and each animal was identified by a unique number. All animals use in the study were euthanized at the end of the study.

Animal Care

The Testing Facility was accredited by the Association for Assessment and Accreditation of Laboratory Animal Care (AAALAC) and licensed by the United States Department of Agriculture (USDA) to conduct research in laboratory animals in compliance with the Animal Welfare Act, USDA regulations and National Research Council (NRC) guidelines (3, 4, 5). Animal activities described herein were subject to review and approval by the Institutional Animal Care and Use Committee (IACUC) of the Testing Facility.

Animal husbandry, diet, water and environmental conditions were performed in compliance with NRC guidelines (17) and Testing Facility standard operating procedures (SOPs).

The materials and methods used in the experiments presented in this example are now described.

Randomization

Animals considered suitable for the study were randomized to treatment groups by body weight and assigned unique consecutive identification numbers within each group. The order in which animals were assigned to undergo procedures was rotated among groups on the basis of identification numbers to minimize procedural bias.

Acclimation to Physical Restraint

Animals were acclimated to the rope-and-collar method of physical restraint and to restraint in a primate chair prior to initiation of treatment.

Tranquilization

Animals were tranquilized (ketamine HCl, 5-10 mg/kg, IM, to effect) as necessary to facilitate handling, blood collection or other technical procedures.

Fasting

All food was withheld overnight prior to tranquilization or anesthesia. Water was not be withheld.

Dose Calculation

Doses were calculated based on Day −1 body weight. The doses were maintained throughout the treatment period.

Dose Administration

All treatments were administered using in-line or syringe-tip low protein-binding filters. IV treatment were administered while animals were restrained in a primate chair, via a percutaneous catheter placed in a peripheral vein, using a clinical grade infusion pump. SC treatment were given in the intrascapular area, using a 23-guage needle.

Blood Collection

Blood samples were collected from tranquilized animals via direct venipuncture of a femoral vein. Blood collection was alternated between left and right femoral veins when possible. Considerable efforts were made to minimize local vascular trauma or bleeding. It was acceptable to not collect individual samples if difficulty in collecting them suggested the likelihood of inducing local vascular trauma (e.g. hematoma formation, arteriopuncture).

Concurrent Therapy

Concurrent therapy in accordance with accepted veterinary practices was utilized if deemed necessary by a veterinarian.

Animal Observations

Body weights were recorded approximately weekly (see Table B). Cage side observations for moribundity and mortality were performed twice daily.

Clinical Observations

Clinical observations for evidence of treatment-related effects were performed beginning prior to and approximately 1 hour after treatment on Day −1, and daily thereafter. On days of SC treatment clinical observations were performed prior to treatment.

Injection Site Observations

The SC injection site (interscapular area) was observed beginning prior to injection on Day 1, and daily thereafter. The site was subjectively scored for swelling and/or erythema (0=none, 1=mild, 2=moderate, 3=marked).

Vital Sign Monitoring During Infusion

During IV treatment vital signs (heart rate, respiratory rate, rectal body temperature and indirect blood pressure) were monitored intermittently for indications of adverse reactions. Representative values for these parameters were recorded prior to, at ˜10 minute intervals during, and at the end of infusion.

If adverse reactions occurred, treatment may have been interrupted or discontinued. A Testing Facility veterinarian determined the appropriate therapy, if any, in consultation with the Study Director and/or study Sponsor's Representative.

Angioplasty and Stenting Procedures

Anticoagulant Therapy

Animals received aspirin (˜40 mg, orally) daily to provide anticoagulant function and minimize stent thrombosis beginning on Day −3.

Antibiotic Therapy

Animals received a single prophylactic injection of benzathine/procaine penicillin-G (42,000 IU/kg, IM) on Day 1 prior to angioplasty.

Anesthesia

Animals were pre-anesthetized (ketamine HCl, 10 mg/kg, IM; atropine SO₄, 0.04 mg/kg, IM) then intubated and maintained in anesthesia with isoflurane inhalant anesthetic gas.

Preparation

Animals were positioned on a procedure table in dorsal recumbency. The bladder was catheterized to prevent urine accumulation. Sites for vascular access were clipped and prepared for aseptic surgery. A catheter was placed in a peripheral vein to facilitate maintenance fluid administration (lactated Ringer's solution, 5-10 mL/kg/hr).

Heparinization

Heparin (100 U/kg, IV, initially) was administered prior to angioplasty to provide anticoagulation. Activated clotting time (ACT) was monitored periodically and additional heparin was administered as necessary to maintain ACT values>250 seconds for the duration of the angioplasty procedure.

Instrumentation

The right carotid artery was surgically exposed and a 6Fr percutaneous vascular introducer sheath (e.g. CP-07711, ARROW International, Reading, Pa. 19605) was placed to facilitate interventional catheter placement.

Utilizing fluoroscopic guidance, a 6Fr guide catheter was passed antegrade to the level at which the distal abdominal aorta bifurcates into the right and left iliac arteries. A radioopaque 0.014-inch guide wire (e.g. 22225M, Advanced Cardiovascular Systems, Inc., Temecula, Calif. 92591) was used to facilitate passage of the guide catheter or other catheters as necessary. Radioopaque contrast media (e.g. Omnipaque™, iohexol injection, Nycomed, Princeton, N.J. 75039) was used as necessary to facilitate fluoroscopy.

Videotaping of Angiography

The fluoroscopic procedures were videotaped for each animal to facilitate measurements for quantitative angiography. Information identifying the study number, study day, animal number and procedure were also recorded on the videotape.

Pre-angioplasty Angiography

Prior to angioplasty, nitroglycerine (50 μg, IA) was administered to induce arterial dilatation. Radioopaque contrast media was administered to facilitate angiography.

Endothelial Denudation via Balloon Angioplasty

n 80 cm, 3Fr Fogarty balloon embolectomy catheter (e.g. 120803F, Baxter Healthcare Corp., Irvine, Calif. 92714) with a balloon appropriately sized for the vessel was passed via the guide catheter into the right iliac artery, to a level ˜4 cm distal to the aortic bifurcation. The balloon was then inflated with 0.6 cc air and withdrawn inflated over an ˜3-cm section of artery to facilitate endothelial denudation. Balloon angioplasty was performed three times. This procedure was then repeated in the contralateral (left) iliac artery and the balloon embolectomy catheter was withdrawn. In some cases the left iliac artery was denuded first, followed by the right.

Stent Placement

An appropriate-sized dilation catheter (Ninja™ PTCA dilation catheter with SLX™ coating, Cordis Corp., Miami Fla. 33102) fitted with a balloon-expandable 7-mm stent (e.g., one half of a 15-mm long stent (e.g. CS15-030, Palmaz-Schatz® crown balloon-expandable stent, Cordis Corp., Miami Fla. 33102)) was then passed into the right iliac artery to the level of the midpoint of endothelial denudation. The balloon was inflated to the appropriate inflation pressure required to expand the stent sufficiently to provide a balloon/stent:artery ratio of 1.1-1.2 (typically 6 Atm for 2.5, 3.0 or 3.5 mm catheters). The balloon was deflated and the catheter was withdrawn. This procedure was repeated in the contralateral (left) iliac artery. In some cases the left iliac artery was stented first, followed by the right.

Post-angioplasty Angiogaphy

Approximately 10 min after placement of the second stent, nitroglycerine (50 μg, IA) was administered to induce arterial dilatation for quantitative angiography of both arteries. Radioopaque contrast media was administered to facilitate angiography.

Recovery

The vascular introducer sheath was removed and the carotid artery was ligated. The incision was closed with appropriate suture. The animals recovered from anesthesia and were returned to their cages.

Analgesia

Animals received a single injection of buprenorphine (0.01 mg/kg, IM) after completion of the procedures.

Follow-up Angiography

Anesthesia

Prior to euthanasia and arterial tissue collection (see Paragraph VIII.L) animals were pre-anesthetized (ketamine HCl, 10 mg/kg, IM; atropine SO₄, 0.04 mg/kg, IM) then intubated and maintained in anesthesia with isoflurane inhalant anesthetic gas.

Preparation

Animals were positioned on a procedure table in dorsal recumbency. A catheter was placed in the peripheral vein. The incision site was clipped and washed; strict asepsis was not be required for this terminal procedure.

Catheter Placement

Heparin (150 U/kg, IV) was administered. Radioopaque contrast media was used as necessary to facilitate fluoroscopy. The left carotid artery was surgically exposed and a 6Fr percutaneous vascular introducer sheath placed. Utilizing fluoroscopic guidance, a 6Fr guide catheter was passed antegrade to the level at which the distal abdominal aorta bifurcates into the right and left iliac arteries. Nitroglycerine (50 μg, IA) was administered. Radioopaque contrast media was administered to facilitate angiography. The fluoroscopic procedures was videotaped for each animal to facilitate measurements for quantitative angiography.

Arterial tissue collection was performed as follows.

Euthanasia

Animals were already anesthetized for follow-up angiography (see Paragraph VIII.K.1). Animals were euthanized in accordance with American Veterinary Medical Association (AVMA) guidelines (6) by deep anesthesia (sodium pentobarbital, 35 mg/kg, IV), followed by exsanguination.

Perfusion

A midline laparotomy incision was made and a cannula was placed in the descending abdominal aorta and advanced to the level of the bifurcation. The iliac arteries were flushed with 100 mL lactated Ringer's solution, followed by perfusion with 0.4% paraformaldeyde (PFA) for 5 min at 100 mm Hg pressure.

Arterial Tissue Removal

Right and left iliac arteries were separately excised, with the proximal ends identified (e.g. by ligature), and immersed in 0.4% PFA.

Limited Gross Necropsy

Animals underwent a limited necropsy, defmed as evaluation of the external body and abdominal and thoracic cavities.

Limited Organ/Tissue Collection

Representative samples from specified organs and tissues (see Table D) were collected and fixed in 10% neutral-buffered formalin for histopathologic evaluation or embedded and frozen in OCT for immunohistology.

TABLE D
Limited Organ/Tissue Collection

| | |
|---|---|
| Injection sites (interscapular area) | Brain (cerebrum) |
| Adrenal glands | Heart |
| Bone marrow (sternum) | Ileum (ileocecocolic junction) |
| Eyes^{a} | Kidney |
| Heart | Liver |
| Kidneys | Lung |
| Large intestine (cecum, colon) | Lymph node (iliac, inguinal) |
| Liver | Sciatic nerve |
| Lung | Spinal cord |
| Lymph nodes (axillary, inguinal, | Spleen |
| mesenteric) | Thymus |
| Small intestine (duodenum, | |
| jejunum, ileum) | |
| Spleen | |
| Thymus | |
| Thyroid gland (with parathyroid) | |

^{a}Eyes were fixed in Davidson's fixative.
^{b}All cell counts were reported as absolute values only. Other cell types (e.g. precursor cells) if observed were counted. Other morphologic features (e.g. RBC staining characteristics) if present, were documented.

Serum Chemistry

Serum samples were analyzed using a chemistry analyzer (see Table F).

TABLE E
Hematology Parameters

| | |
|---|---|
| Total leukocyte count (WBC) | Blood smear evaluation and |
| Erythrocyte count (RBC) | differential:^{b} |
| Hemoglobin concentration (HGB) | Segmented neutrophil count |
| Hematocrit value (HCT)^{a} | (APLY) |
| Mean corpuscular volume (MCV)^{a} | Band neutrophil count (ABND) |
| Mean corpuscular hemoglobin (MCH)^{a} | Lymphocyte count (ALYM) |
| Mean corpuscular hemoglobin | Monocyte count (AMNO) |
| concentration (MCHC)^{a} | Eosinophil count (AEOS) |
| Platelet count (PLT) | Basophil count (ABSO) |
| | Nucleated RBC count (ANRC) |

^{a}= Calculated value.
^{b}= All cell counts were reported as absolute values only. Other cell types (e.g. precursor cells), if observed, were counted. Other morphologic features (e.g. RBC staining characteristics) if present, were documented.

TABLE F
Serum Chemistry Parameters

| | |
|---|---|
| Glucose (GLU) | Sodium (NA) |
| Blood urea nitrogen | Potassium (K) |
| (BUN) | Chloride (CL) |
| Creatinine (CRE) | Total cholesterol (CHOL) |
| Total protein (TPR) | Total bilirubin (TBIL) |
| Albumin (ALB) | Triglycerides (TRG) |
| Globulin (GLOB)^{a} | Alanine aminotransferase (ALT) |
| Albumin/Globulin ratio | Aspartate aminotransferase (AST) |
| (A/G)a | Alkaline phosphatase (ALK) |
| Calcium (CAL) | |
| Phosphorus (PHOS) | |
| Gamma glutamyl | |
| transferase (GGT) | |

a = Calculated value.

Samples for Additional Analyses

Blood samples for pharnacodynamic assays and sera samples for pharmacokinetic and immunogenicity assays were obtained.

Pharmacokinetics

Serum therapeutic 1B4 monoclonal antibody (mAb) levels were determined by enzyme-linked immunosorbent assay (ELISA) for murine IgG. Briefly, 96-well plates (NUNC #4-39454) were coated with 100 μl goat-anti-mouse IgG+IgM antibody (Jackson Immunoresearch #115-005-068) at 2.5 μg/ml in carbonate buffer pH 9.3 overnight at 4° C. Plates were subsequently washed 3 times with PBS 0.5% Tween-20 and blocked with 300 μl PBS/1% BSA for 60 minutes at 37° C. Following 3 additional washes with PBS-Tween, serum samples were diluted 1:100 in PBS/1%BSA and 100 μl aliquots were added to duplicate wells in the plate. The antibody standard (MOPC-21, Sigma) was diluted to 50 ng/ml and 100 μl aliquots were added to the plate. Subsequently, all samples were diluted 2-fold across the plate and incubated at room temperature for 2 hours. The plate was subsequently washed again with PBS/0.5%Tween-20 and 100 μl of peroxidase-conjugated goat anti-mouse IgG+IgM (Jackson Immunoresearch #115-035-068) was added at a concentration of 375 ng/ml and incubated for 2 hours at room temperature. Following additional washes with PBS-Tween, plates were developed with OPD (Sigma) in citric acid buffer pH 5.0, and analyzed on a 96-well fluorescent plate reader (Dynatech MR4000) at 492 nm. The dilutions of the antibody standard was used to construct a standard curve, and the serum antibody concentration was automatically derived from the standard curve and dilution factor data provided using Biolinx 2.22 software.

Pharmacodynamics

Target Saturation

Saturation of 1B4 target (CD18) on appropriate leukocyte subsets (neutrophils and monocytes) was determined by flow cytometry assays.

Determination of Saturation of Circulating Leukocvtes with 1B4 (anti-CD18)

Blood was collected in heparin from the test animals at specified intervals prior to and after the administration of 1B4. Samples of whole blood were stained (“spiked”) with supersaturating amounts of 1B4 or nothing. The blood samples were washed in buffer and stained with FITC conjugated goat-anti-mouse IgG. After daily standardization of the flow cytometer with FITC-labeled beads, to ensure equivalent day-to-day sensitivity to FITC, the blood was lysed using ammonium chloride lysing solution and the fluorescence of lymphocyte, monocyte and granulocyte populations was determined. The degree of saturation of CD18 on either neutrophils or monocytes by the administered 1B4 was determined by the difference between the mean channel fluorescence (MCF) of the sample with no added 1B4 and the sample with the added spike of 1B4. In practice, free CD18 on the surface of the cells which was not coated with the 1B4 delivered in vivo was stained by the exogenously added 1B4 and the mean channel fluorescence of the unspiked sample was dimmer than the mean channel fluorescence of the spiked sample. The difference in staining intensity was a reflection of free (unsaturated) CD18 on the cell surface.

Determination of Saturation of Circulating Leukocytes with S-S.1 (Irrelevant Isotype Control Antibody)

S-S.1 is a non-cell binding irrelevant murine antibody. Assays to determine potential “saturation” of leukocyte antigens with this mAb were performed as above, with the understanding that a positive result (cell staining) was unlikely to be seen and that there would consistently be no difference in mean channel fluorescence between unspiked and spiked samples over time.

Peripheral Blood Leukocyte Dynamics

The effect of mAb administration on leukocyte dynamics (trafficking, margination/demargination) was identified indirectly by evaluating the numbers of leukocytes in circulation, as compared to prior to treatment. Inhibition of leukocyte adhesion and/or chemotaxis would be expected to prevent normal trafficking and to result in elevated circulating cell numbers. Routine hematology was performed to determine the total numbers of peripheral blood leukocytes, as well as the number of neutrophils, lymphocytes and monocytes.

Immunogenicity

Measurement of Antibody Responses to 1B4 (CD18)

Sera samples were collected at specified times and stored frozen until completion of the study. Anti-1B4 antibodies were detected using two assays.

The first assay was designed to detect both anti-idiotype and anti-isotype antibodies. This assay was performed by coating the wells of a microtiter plate with 1B4 and blocking unused protein binding sites with BSA. The sera was then diluted appropriately and several dilutions were added to duplicate wells of the plate. Antibodies in the sera were allowed to bind for 2 hours at 37 degrees and then the wells were shaken out and washed 3 times in PBS with Tween 20. Monkey anti-1B4 antibodies were detected with HRP-conjugated goat anti-human IgG (absorbed against mouse proteins). After 2 hours, unbound detection antibody was washed away in three washes of the plate with PBS Tween. Bound complexes were detected by the addition of o-phenylenediamine to produce a yellow color. Color was read at 490 nm on an ELISA plate reader. Titers were determined by calculating the inverse of the dilution of the sera which produced an optical density equivalent to the optical density produced by a specific dilution of a commercial HRP-conjugated goat anti-mouse IgG (absorbed against human serum proteins).

The second assay was used to assess the proportion of the response which reacted with the 1B4 idiotype compared with the response to mouse IgG2a. This was a competitive ELISA in which the sera from a peak antibody response sample was diluted to produce an optical density between 0.6-1.0. The diluted sera was added to triplicate wells of an ELISA plate coated with 1B4 as above. The sera was added alone, mixed with 5 μg of commercial mouse IgG2a, or mixed with 5 μg of 1B4. The ELISA was carried out as above and monkey antibody bound to the 1B4 on the plate was detected using HRP-anti-human IgG, as above. By comparing the optical density of signals produced by the uncompeted sera with those produced by sera spiked with mouse IgG2a or 1B4 it was possible to assess the specificity of the anti-1B4 antibodies which developed in animals treated with 1B4.

Measurement of Antibody Responses to S-S.1 (Irrelevant Isotype Control Antibody)

Anti-S-S.1 antibodies were detected using two assays as described above.

Quantitative Angiography Calculations:

Control of Bias

At the time of angioplasty and stenting angiography measurements were performed. The measurement were taken in a non-blinded fashion to determine the diameter of each artery and to select the appropriate size balloon dilation catheter and inflation pressure for expansion of the stents, thus providing the desired balloon/stent:artery ratio. Non-blinded measurements will also be performed at follow-up. For the purpose of evaluating treatment effect(s), recorded video images were replayed on a larger video screen and evaluated in a blinded fashion by an independent observer.

Angiography Measurements

Blinded angiography measurements were performed by measuring the fluoroscopy images directly from the video screen at the mid-stent area with digital calipers. For both iliac arteries, the following parameters were measured (in mm):

Angioplasty/stenting

Actual guide catheter o.d. (actual measurement) (a)

Observed guide catheter o.d. (observed on video screen as magnified image) (b)

Pre-angioplasty luminal i.d. (x)

Post-angioplasty in-stent inflated balloon o.d. (y)

Post-angioplasty/stent in-stent luminal i.d. (x′)

Follow-up

Actual follow-up guide catheter o.d. (c)

Observed follow-up guide catheter o.d. (d)

Follow-up in-stent luminal i.d. (x″)

Restenosis Calculations

The following calculations were performed:

Angioplasty/stenting

Magnification correction factor 1 (MCF1)=[b]÷[a]

Balloon/stent:artery ratio=[y:x]=1.1-1.2, ideally

Acute luminal gain (ALG; in mm)=[(x′)(MCF1)]−[(x)(MCF1)]

Follow-up

Magnification correction factor 2 (MF2)=[d]÷[c]

Late luminal loss (LLL; in mm)=[(x′)(MCF1)]−[(x″)(MCF2)]

Arterial Tissue Analysis

Control of bias

Arterial tissue samples were randomly assigned accession or identification numbers that do not indicate group or animal number. The person(s) evaluating arterial tissue samples for effect(s) of treatment were blinded to the identity of the samples.

Tissue Processing

The non-stented (balloon-injured) proximal and distal arterial segments were separated from the stented segments, with the proximal ends of each was identified and marked. Stented arterial segments were embedded in methacrylate and multiple 5 mm cross-sections were cut with a tungsten carbide knife. Non-stented arterial segments were embedded in paraffin to preserve antigenicity, but were not processed further unless warranted.

Stented sections were stained with verHoeff's tissue elastin stain, hematoxylin and eosin (H+E), and various immunocytochemical markers for cells incorporating BrdUrd or for cell types such as smooth muscle cells, endothelial cells, and inflammatory cells.

Evaluation of Neointimal Hyperplasia

In-stent cross-sectional neointimal (on the luminal side of the internal elastic membrane [IEL]) and medial (on the abluminal side of the IEL) areas (mm²) were measured histomorphometrically using computer-assisted digital planimetry (3). To minimize sampling error, 3 elastin-stained in-stent cross-sections, one each from the proximal middle and distal portions of the right and left iliac arteries, were analyzed morphometrically. The composite value for the left or right artery was expressed as the mean value of the 3 measurements for each artery.

Each cross-section was scored (0-3) for the deep stent-induced arterial injury associated with each stent strut (8-12/cross-section) and an average depth of injury score for each cross-section was calculated (19). These values were used to evaluate whether the initial injury was comparable across groups.

Statistical Analysis

Analysis of efficacy data by T-test between treated and control groups was performed and these values are reported.

The results of the experiments presented in this example are now described.

Safety

There were no treatment-related effects on vital signs during infusion. There were no treatment-related effects on body weight or clinical observations during the study. Individual injection sites in one or more animals showed, mild, transient erythema which was not considered an adverse reaction. There were no adverse events associated with the catheterization incisions (i.e., no impairment of wound healing and no indication of bacterial infection). There were no adverse effects on clinical pathology parameters. As expected, serum globulin levels were elevated in treated and control animals. Leukocyte counts were affected by 1B4 administration (see below). There were no treatment-related gross lesions at necropsy.

Pharmacokinetics

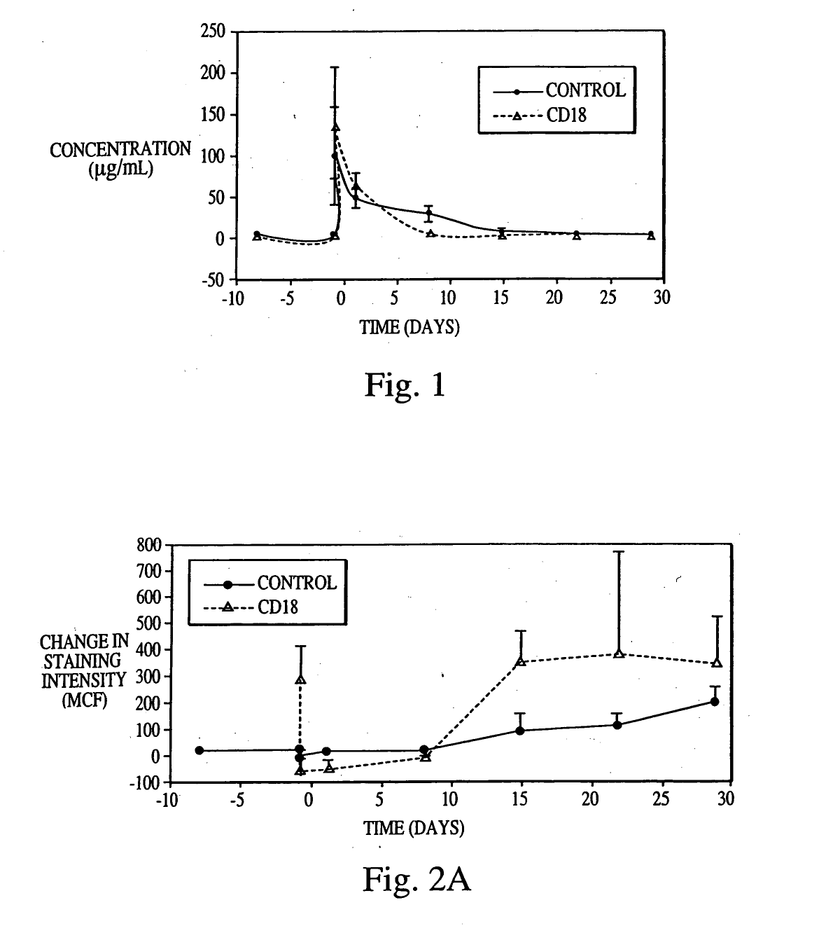

Serum mAb levels (mean±stdev), relative to control mAb, are presented in FIG. 1.

Administration of 1B4 resulted in serum concentrations >50 μg/mL at the time of angioplasty and stent deployment (Day 1) and maintenance of serum concentrations>1 μ/mL through Day 8. By Day 15, 1B4 levels were virtually undetectable, despite continuation of dosing from Day ⁻1 to 13.

Pharmacodynamics

Leukocyte Target Saturation

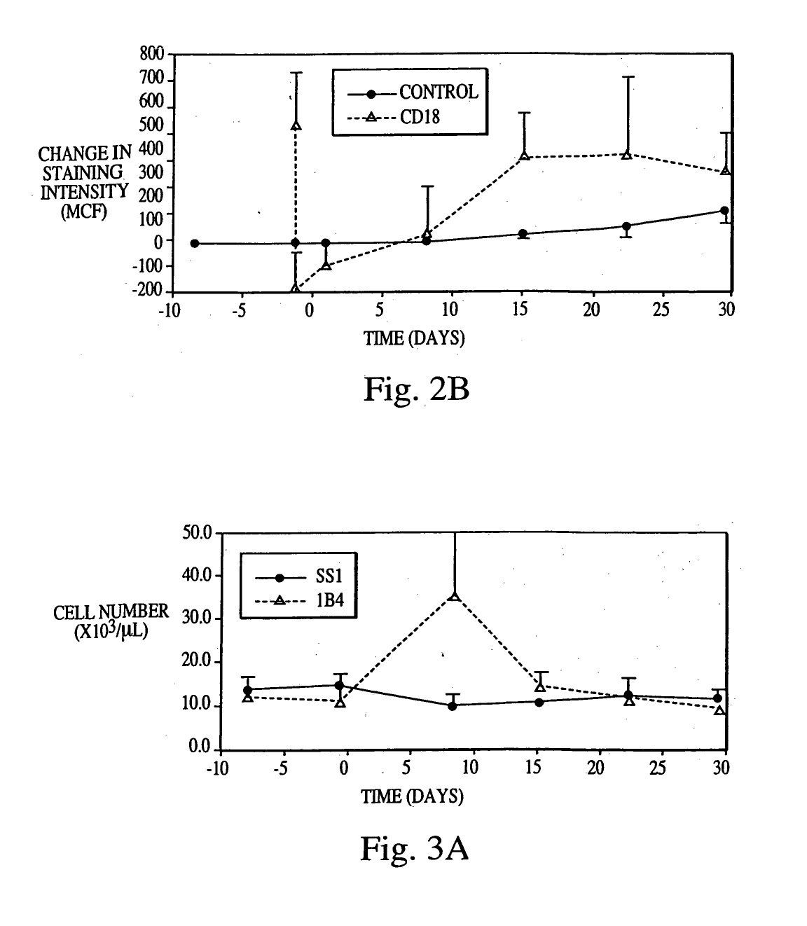

Leukocyte target saturation (mean±stdev), relative to control mAb, is presented in FIG. 2.

Administration of 1B4 resulted in rapid saturation of neutrophil and monocyte CD18 on Day ⁻1 immediately after IV infusion and maintenance of target saturation through Day 8. By Day 15, available CD18 binding sites on leukocytes (unsaturated targets) returned to baseline levels.

Peripheral Blood Leukocyte Dynamics

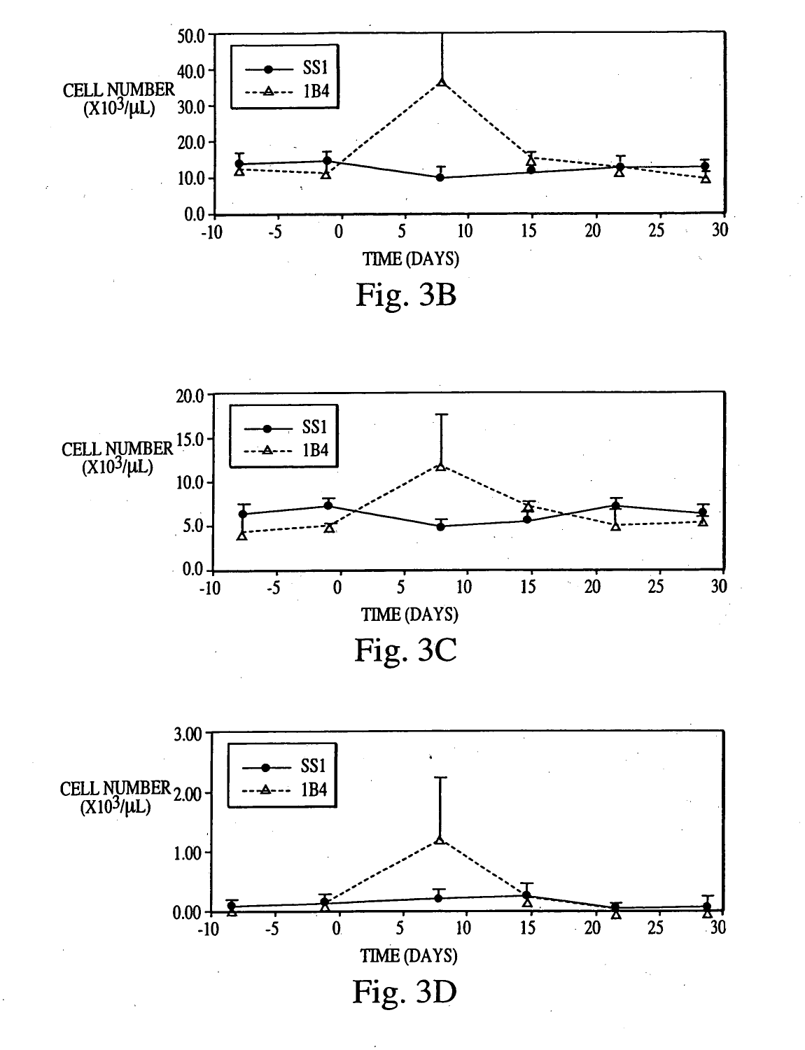

Peripheral blood leukocyte counts (mean±stdev), relative to control mAb, are presented in FIG. 3.

Administration of 1B4 resulted in altered leukocyte dynamics attributed to CD18 saturation, as indicated by the pronounced leukocytosis, neutrophilia, lymphocytosis and monocytosis on Day 8. Although not determined, these cell counts were likely elevated at earlier time points as well.

Immunogencity

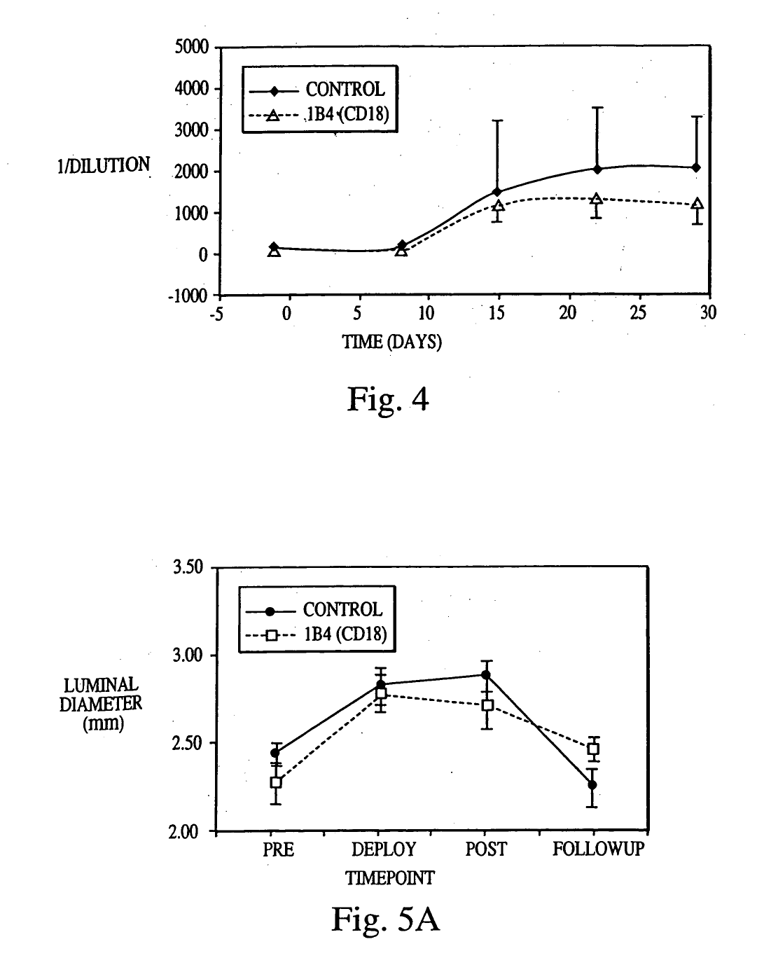

The anti-mAb antibody titers (mean±stdev), relative to control mAb, are presented in FIG. 4.

Anti-globulin responses developed in all animals, detected as early as Day 8. The majority of these responses were anti-idiotype (directed against the variable region, specifically the complementarity determining region), rather than anti-isotype (directed against the constant region). The rapid increase in potentially neutralizing anti-idiotype antibodies from Day 8 to Day 15 corresponds with the loss of circulating mAb levels, the loss of leukocyte target saturation and the return of peripheral blood leukocyte counts to baseline (normal) levels. These observations are consistent with anti-mAb antibodies binding to the therapeutic mAb and preventing (neutralizing) the activity. Further, these observations suggest that effective sera/leukocyte levels of therapeutic mAb were only maintained through Day 8.

Efficacy

Quantitative Angiography

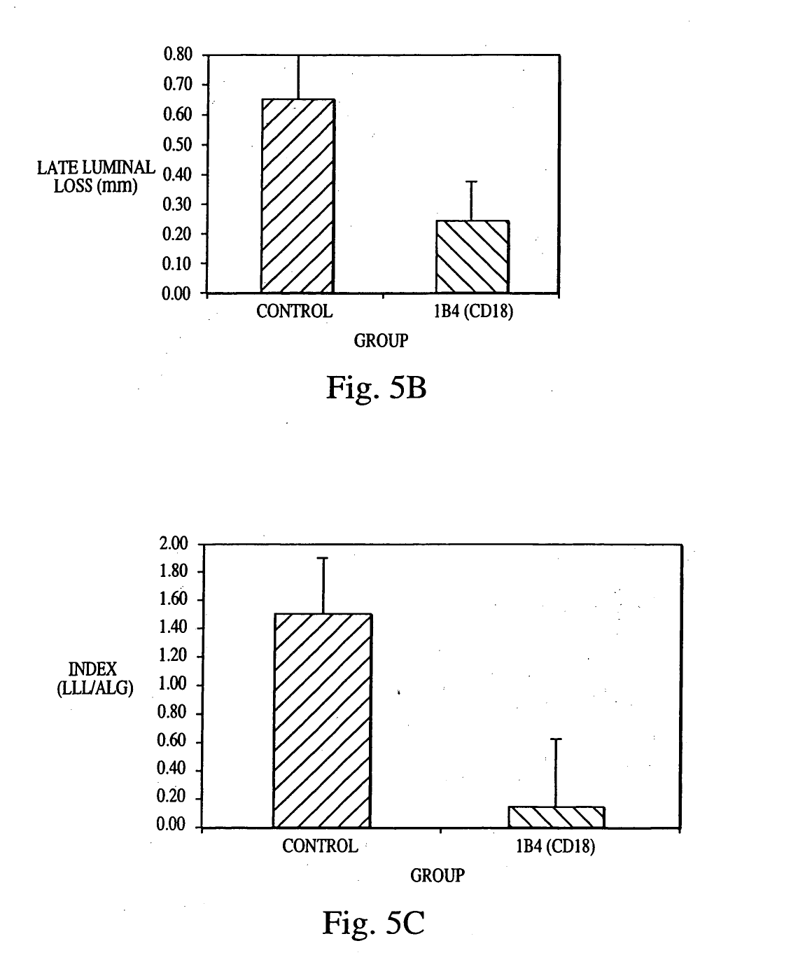

The blinded quantitative angiography results (mean±stdev), relative to control mAb, are presented in FIG. 5.

Administration of 1B4 tended to decrease the late luminal loss (LLL) (p=0.06) and significantly decreased the index (LLL/ALG) (p<0.05) as measured at the mid-stent region of the iliac arteries.

Histomorphometric Analysis

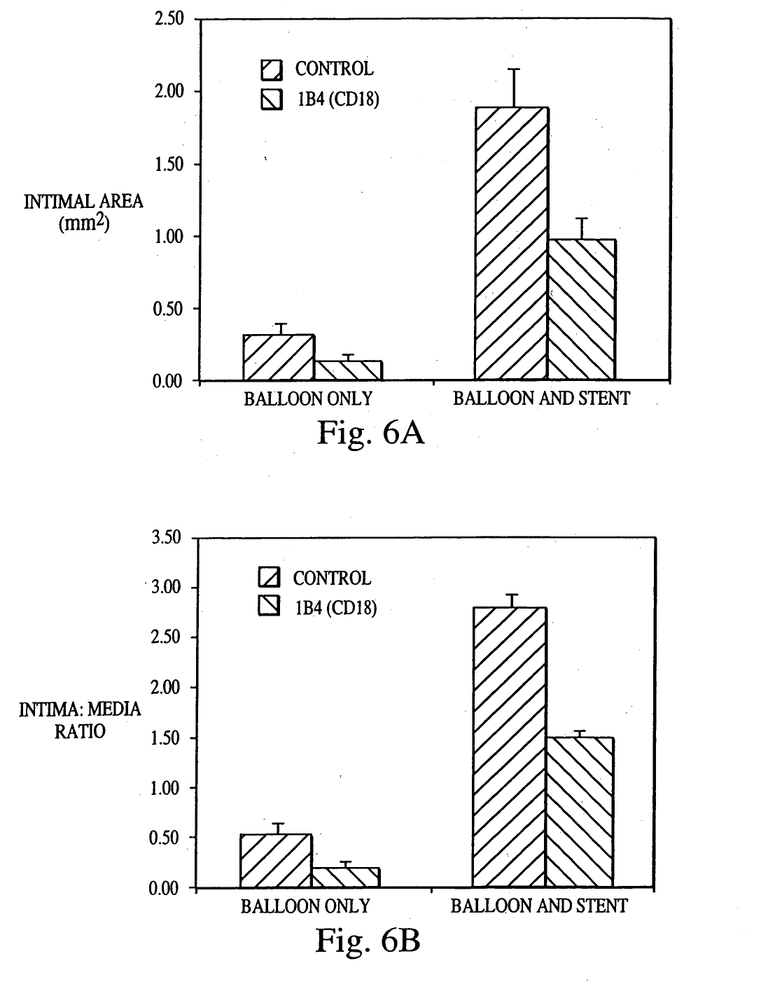

The blinded histomorphometric analysis results (mean±stdev), relative to control mAb, are presented in FIG. 6.

Severity scores indicated there was no difference between groups in the degree of stent-mediated injury to the arteries, thus differences between groups are attributable to treatment and not to differences in degree of injury.

Administration of 1B4 inhibited neointimal hyperplasia within the balloon-only (p=0.02 for intimal area, p=0.01 for I:M ratio) and balloon+stent (p<0.01 for both intimal area and I:M ratio) segments of the iliac arteries. Because CD18 is present primarily on neutrophils, and to a lesser extent on mononuclear cells (monocytes and lymphocytes), this data suggests that neutrophils are important (and perhaps predominant) contributors to both balloon-only and balloon+stent neointimal hyperplasia. It does not exclude the possibility that other cells (i.e., mononuclear cells) expressing CD18 also are contributors to neointimal hyperplasia with either injury. The observation of effective reduction of balloon-only and balloon+stent neointimal hyperplasia with anti-CD18 inhibition may be relevant for balloon-only and balloon+stent (in-stent) restenosis in humans.

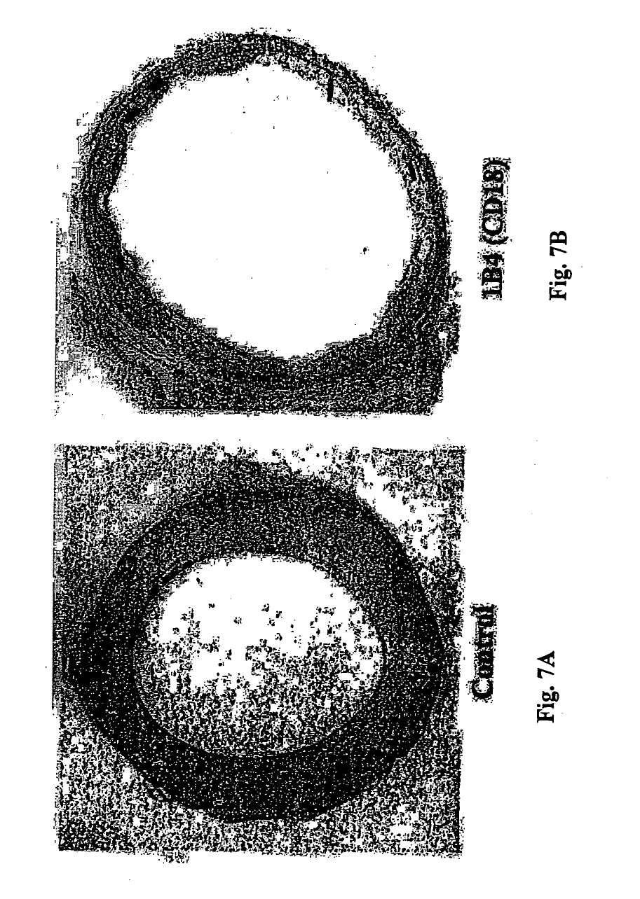

The effect of CD18 blockade on neointimal hyperplasia is illustrated in FIG. 7.

The references cited in this example include the following, numbered as indicated in this example.

1. Code of Federal Regulations (CFR). Title 21; Part 58, Good Laboratory Practice Regulations: Final Rule. Washington (DC), Office of the Federal Register. Dec. 22, 1978 (Revised Apr. 1, 1993).

2. Holmes D R, Vlietstra R E, Smith H C, Vetrovec G W, Kent K M, Cowley M J, Faxon D P, Gruntzig A R, Kelsey S F, Detre K M, van Raden M J, Mock M B. Restenosis after percutaneous transluminal angioplasty (PTCA): a report from the PCTS registry from the National Heart, Lung and Blood Institute. Am J Cardiol 1984;53:77C-81C.

3. Serruys P W, Luitjen H E, Beatt K J, Geuskens R, de Feyter P J, van den Brand M, Reiber J H, ten Katen H J, van Es G A, Hugenholtz P G. Incidence of restenosis after successful coronary angioplasty: a time-related phenomenon: a quantitative angiographic study in 342 consecutive patients at 1, 2, 3, and 4 months. Circulation 1988;77:361-371.

4. Rogers C, Edelman E R, Simon D I. A mAb to the β2-leukocyte integrin Mac-1 (CD11b/CD18) reduces intimal thickening after angioplasty or stent implantation in rabbits. Proc Natl Acad Sci USA 1998;95:10134-10139.

5. Rogers C, Edelman E R: Endovascular stent design dictates experimental restenosis and thrombosis. Circulation 1995;91:2995-3001.

6. Ponath P. Chemokine receptor antagonists: novel therapeutics for inflammation and AIDS. Exp Opin Invest Drugs 1998;7:1-18.

7. Nelken N A, Coughlin S R, Gordon D, Wilcox J N. Monocyte chemoattractant protein-1 in human atheromatous plaques. J Clin Invest 1991;88:1121-1127.

8. Boring L, Gosling J, Cleary M, Charo I F. Decreased lesion formation in CCR2−/−mice reveals a role for chemokines in the initiation of atherosclerosis. Nature 1998;394:894-897.

9. Gu L, Okada Y, Clinton S K, Gerard C, Sukhova G K, Libby P, Rollins B J. Absence of monocyte chemoattractant protein-1 reduces atherosclerosis in low density lipoprotein receptor-deficient mice. Mol Cell 1998;2:275-281.

10. Furukawa Y, Matsumori A, Ohashi N, Shioi T, Ono K, Harada A, Matsushima K, Sasayama S. Anti-monocyte chemoattractant protein-1/monocyte chemotactant and activating factor antibody inhibits neointimal hyperplasia in injured rat carotid arteries. Circ Res 1999;84:306-314.

11. Kling D, Fingerle J, Harlan J M, Lobb R R, Lang F. Mononuclear leukocytes invade rabbit arterial intima during thickening formation via CD 18- and VLA-4-dependent mechanisms and stimulate smooth muscle migration. Circ Res 1995;77:1121-8.

12. Kling D, Fingerle J, Harlan J M. Inhibition of leukocyte extravasation with a monoclonal antibody to CD18 during formation of experimental intimal thickening in rabbit carotid arteries. Arterioscler Thromb 1992;12:997-1007.

13. Golino P, Ambrosio G, Ragni M, Cirillo P, Esposito N, Willerson J T, Rothlein R, Petrucci L, Condorelli M, Chiariello M, Buja L M. Inhibition of leukocyte and platelet adhesion reduces neointimal hyperplasia after arterial injury. Thromb Haemostasis 1997;77:783-8.

14. Guzman L A, Forudi F, Villa A E, Topol E J. Role of leukocytes in neointimal formation after balloon angioplasty in the rabbit atherosclerotic model. Coronary Art Dis 1995;6:693-701.

15. United States Code. Title 7 U.S.C. Sections 2131-22159 (The Animal Welfare Act as amended by P. L. 99-198), effective Dec. 23, 1986.

16. Code of Federal Regulations (CFR). Title 9; Chapter 1, Subchapter A (Animal Welfare Standards), Final Rule, Parts 1-3. Washington (DC), Office of the Federal Register. Jan. 1, 1997.

17. National Research Council, Institute of Laboratory Animal Resources. Guide for the Care and Use of Laboratory Animals. Washington (DC): National Academy Press, 1996.

18. American Veterinary Medical Association. Report of the American Veterinary Association (AVMA) panel on euthanasia. J Am Vet Med Assoc 1993;202:229-249.

19. Schwartz R S, et al., Restenosis and Proportional Neointimal Response to Coronary Artery Injury: Results in a Porcine Model. J Am Coll Cardiol 1992;19:267-274.

The disclosure of every patent, patent application, and publication cited herein is hereby incorporated herein by reference in its entirety.

While this invention has been disclosed with reference to specific embodiments, it is apparent that other embodiments and variations of this invention can be devised by others skilled in the art without departing from the true spirit and scope of the invention. The appended claims include all such embodiments and equivalent variations.

What is claimed is:

1. A method of inhibiting stenosis in a human blood vessel, the method comprising administering to the human an anti-CD18 antibody which binds specifically with at least the CD18 portion of a mammalian protein which comprises CD18, whereby stenosis is inhibited in the vessel.

2. The method of claim 1, wherein the anti-CD18 antibody binds specifically with substantially only the CD18 portion of the protein.

3. The method of claim 1, wherein the anti-CD18 antibody has an epitopic specificity which is the same as or similar to that of monoclonal antibody 1B4.

4. The method of claim 1, wherein the anti-CD18 antibody is monoclonal antibody 1B4.

5. The method of claim 1, wherein the anti-CD18 antibody binds specifically with at least the CD18 portion of a primate protein which comprises CD18.

6. The method of claim 1, wherein the protein is a leukocyte cell-surface antigen.

7. The method of claim 6, wherein the antigen is selected from the group consisting of Mac-1, LFA-1, p150,95, and CD11d/CD18.

8. The method of claim 7, wherein the antigen is Mac-1.

9. The method of claim 6, wherein binding of the anti-CD18 antibody with the antigen inhibits binding of a natural ligand of the antigen therewith.

10. The method of claim 9, wherein the ligand is selected from the group consisting of ICAM-1, ICAM-2, ICAM-3, C3bi, factor X, fibrin, and fibrinogen.

11. The method of claim 10, wherein the ligand is selected from the group consisting of ICAM-1, C3bi, factor X, fibrin, and fibrinogen.

12. The method of claim 1, wherein binding of the anti-CD18 antibody with the protein modulates at least one function normally associated with binding of a natural ligand of the protein therewith.

13. The method of claim 12, wherein the function is selected from the group consisting of binding of leukocytes with vascular endothelium, translocation of leukocytes through vascular endothelium, infiltration of leukocytes into intimal vascular tissue, release of a chemotactic factor from leukocytes in a vascular tissue, release of a growth factor from leukocytes in a vascular tissue, leukocyte-binding-associated release of a chemotactic factor from a vascular tissue, and leukocyte-binding-associated release of a growth factor from a vascular tissue.

14. The method of claim 13, wherein the leukocytes are neutrophils.

15. The method of claim 1, wherein the blood vessel is a vessel in which the vascular endothelium has been traumatically perturbed.

16. The method of claim 15, wherein the blood vessel is selected from the group consisting of a grafted blood vessel, a blood vessel in which an angioplasty balloon has been inflated, a blood vessel comprising a portion at which a laser angioplasty procedure has been performed, a blood vessel which has sustained a crushing injury, and a blood vessel into which a stent has been placed.

17. The method of claim 1, wherein the blood vessel is a vessel in which the vascular endothelium has non-traumatically deteriorated.

18. The method of claim 17, wherein the blood vessel is selected from the group consisting of an atherosclerotic blood vessel and an arteriosclerotic blood vessel.

19. The method of claim 1, wherein the blood vessel is selected from the group consisting of a coronary blood vessel and a cerebral blood vessel.

20. The method of claim 1, wherein the anti-CD18 antibody is a whole antibody.

21. The method of claim 1, wherein the anti-CD18 antibody is an antibody fragment.

22. The method of claim 21, wherein the antibody fragment is selected from the group consisting of Fv, Fab, Fab′, and F(abN)₂ fragments.

23. The method of claim 1, wherein the anti-CD18 antibody is a chimeric antibody.

24. The method of claim 1, wherein the anti-CD18 antibody is a humanized antibody.

25. The method of claim 1, wherein the anti-CD18 antibody is a human antibody.

26. The method of claim 1, wherein the anti-CD18 antibody is administered to the human by providing the anti-CD18 antibody to the blood vessel.

27. The method of claim 26, wherein the anti-CD18 antibody is provided to the vessel prior to traumatically perturbing the endotheliurn of the vessel.

28. The method of claim 26, wherein the anti-CD18 antibody is provided to the vessel after traumatically perturbing the endothelium of the vessel.

29. The method of claim 1, wherein the stenosis is restenosis following an angioplastic intervention performed upon the human.

30. The method of claim 29, wherein the intervention is a balloon angioplastic intervention.

31. The method of claim 29, wherein the intervention is emplacement of a vascular stent within the vessel.

32. The method of claim 1, wherein the mammalian protein is a human protein.

33. A method of alleviating stenosis in a human blood vessel, the method comprising administering an antibody to the vessel, wherein the antibody binds specifically with at least the CD18 portion of a mammalian protein which comprises CD18, whereby stenosis is alleviated in the vessel.

34. A kit for assessing stenosis in a human blood vessel, the kit comprising an anti-CD18 antibody having a detectable label and an instructional material which describes detecting the anti-CD18 antibody in a blood vessel of the human.

35. The kit of claim 34, wherein the detectable label is a gamma radiation source.

36. A method of inhibiting stenosis in a human blood vessel, the method comprising removing leukocytes which bind specifically with an anti-CD18 antibody from the human's blood, whereby stenosis is inhibited in the vessel

37. A method of alleviating stenosis in a human blood vessel, the method comprising removing leukocytes which bind specifically with an anti-CD18 antibody from the human's blood, whereby stenosis is alleviated in the vessel

38. A method of inhibiting interaction of a leukocyte having a CD 18-containing cell-surface protein with vascular endothelium in a human, the method comprising contacting the leukocyte with an anti-CD18 antibody, whereby interaction of the leukocyte with vascular endothelium is inhibited.

39. The method of claim 38, wherein the leukocyte is selected from the group consisting of lymphocytes, monocytes, granulocytes, neutrophils, T cells, and basophils.

40. The method of claim 39, wherein the leukocyte is a neutrophil.

41. The method of claim 38, wherein the interaction of the leukocyte with the vascular endothelium is binding of the leukocyte with the vascular endothelium.

42. The method of claim 38, wherein the interaction of the leukocyte with the vascular endothelium is translocation of the leukocyte across the vascular endothelium.

43. A method of assessing the presence of leukocytes associated with vascular stenosis in blood obtained from a human, the method comprising
contacting the blood with an anti-CD18 antibody and
detecting binding of the anti-CD18 antibody with leukocytes in the blood, wherein binding of the anti-CD18 antibody with leukocytes in the blood is an indication of the presence of leukocytes associated with vascular stenosis in the blood.

44. The method of claim 43, wherein binding of the anti-CD18 antibody with leukocytes in the blood is quantified.

45. The method of claim 43, wherein binding of the anti-CD18 antibody with leukocytes in the blood of the human is compared with binding of the anti-CD18 antibody with leukocytes in control blood obtained from a human selected from the group consisting of a human afflicted with vascular stenosis and a human not afflicted with vascular stenosis.

46. A kit for assessing the presence of leukocytes associated with vascular stenosis in blood obtained from a human, the kit comprising
i) an anti-CD18 antibody and
ii) an instructional material which describes at least one of
a) quantifying the presence of the leukocytes in the blood of the human,
b) the content of the leukocyte in the blood of a human afflicted with vascular stenosis, and
c) the content of the leukocyte in the blood of a human not afflicted with vascular stenosis.

47. A method of inhibiting a disorder associated with stenosis in a blood vessel of a human, the method comprising administering to the human an anti-CD18 antibody which binds specifically with at least the CD18 portion of a mammalian protein which comprises CD18, whereby stenosis is inhibited in the vessel and the disorder is thereby inhibited.

48. A method of alleviating a disorder associated with stenosis in a blood vessel of a human, the method comprising administering to the human an anti-CD18 antibody which binds specifically with at least the CD18 portion of a mammalian protein which comprises CD18, whereby stenosis is alleviated in the vessel and the disorder is thereby alleviated.
